(19) United States
(12) Patent Application Publication
(10) Pub. No.: US 2026/0091236 A1
(43) Pub. Date: Apr. 2, 2026

(54) MEDICAL DEVICE AND METHOD FOR DETERMINING RISK OF A CARDIAC EVENT

(71) Applicant: Medtronic, Inc., Minneapolis, MN (US)

(72) Inventors: Alfonso ARANDA HERNANDEZ, Shoreview, MN (US); Paul J. DEGROOT, Minneapolis, MN (US)

(21) Appl. No.: 19/112,241

(22) PCT Filed: Sep. 13, 2023
(86) PCT No.: PCT/IB2023/059099

§ 371 Date: Mar. 14, 2025

Related U.S. Application Data
(60) Provisional application No. 63/377,227, filed on Sep. 27, 2022.

(51) Int. Cl. A61N 1/365; G16H 50/30

(52) U.S. Cl. CPC A61N 1/365; G16H 50/30

(57) ABSTRACT

A medical device is configured to receive up to two cardiac electrical signals. For each cardiac cycle of multiple cardiac cycles, the device may derive a T-wave loop in at least two dimensions using one or two of the up to two cardiac electrical signals. The medical device may determine a repolarization measurement representative of each T-wave loop and determine a change in the repolarization measurement from a previously determined repolarization measurement. The device may determine a metric of the determined changes in the repolarization measurements.

This application claims the benefit of U.S. Provisional Patent Application Ser. No. 63/377,227, filed Sep. 27, 2022, the entire content of which is incorporated herein by reference.

### TECHNICAL FIELD

The disclosure relates generally to a medical device and method for determining a metric of changes in cardiac repolarization that is indicative of the risk of a cardiac event.

### BACKGROUND

Medical devices may sense electrophysiological signals from the heart, brain, nerve, muscle or other tissue. Such devices may be implantable, wearable or external devices using implantable and/or surface (skin) electrodes for sensing the electrophysiological signals. In some cases, such devices may be configured to deliver a therapy based on the sensed electrophysiological signals. For example, implantable or external cardiac pacemakers, cardioverter defibrillators, cardiac monitors and the like, sense cardiac electrical signals from a patient's heart. The medical device may sense cardiac electrical signals from a heart chamber and deliver electrical stimulation therapies to the heart chamber using electrodes carried by a medical electrical lead that positions electrodes within or on the patient's heart to promote a normal heart rhythm.

During normal sinus rhythm (NSR), the heartbeat is regulated by electrical signals produced by the sino-atrial (SA) node located in the right atrial wall. Each depolarization signal produced by the SA node spreads across the atria, causing the depolarization and contraction of the atria, and arrives at the atrioventricular (AV) node. The AV node responds by propagating a depolarization signal through the bundle of His of the atrioventricular septum and thereafter to the bundle branches and the Purkinje muscle fibers of the right and left ventricles, sometimes referred to as the “His-Purkinje system.” Depolarization of the atrial tissue can be observed as P-waves in an electrocardiogram (ECG). Depolarization of the ventricular tissue can be observed as R-waves in an ECG. Repolarization of the ventricular myocardium following depolarization is represented by the T-wave in cardiac electrical signals. Variations in repolarization of the myocardium may be related to changes in sympathetic nervous system activity and have been proposed to be related to risk of sudden cardiac death.

### SUMMARY

In general, this disclosure is directed to a medical device and techniques for sensing up to two cardiac electrical signals and determining a metric of repolarization changes of the heart for assessing a patient's risk of a cardiac event, such as arrhythmia, myocardial infarct, or sudden cardiac death. Processing circuity of the medical device is configured to determine a repolarization measurement from each of multiple cardiac cycles. The medical device may determine the repolarization measurement from the T-waves of one cardiac electrical signal or from the T-waves of two cardiac electrical signals. The repolarization measurement may be determined by deriving a T-wave loop in two dimensions or in three dimensions from the one or two cardiac electrical signals. The repolarization measurement may be determined by the processing circuitry by determining a T-wave vector representative of the T-wave loop. The processing circuitry may determine changes between successive repolarization measurements, e.g., between successive, T-wave vectors and determine a metric of the determined changes as an indicator of risk of a cardiac event.

In one example, the disclosure provides a medical device comprising processing circuitry configured to receive up to two cardiac electrical signals. The processing circuitry can be configured to, for each of a plurality of cardiac cycles of the received cardiac electrical signal(s) consisting of the up to the two cardiac electrical signals, derive a T-wave loop in at least two dimensions, determine a repolarization measurement representative of the T-wave loop; and determine a change in the repolarization measurement from a previously determined repolarization measurement. The processing circuitry may determine a metric of the determined changes in the repolarization measurements and determine when the metric is greater than a risk threshold associated with a cardiac event. The medical device may include a telemetry circuit configured to transmit a risk notification in response to the metric being greater than the risk threshold.

In another example, the disclosure provides a method performed by a medical device that includes receiving up to two cardiac electrical signals. The method can include, for each of a plurality of cardiac cycles of the received cardiac electrical signal(s) consisting of the up to the two cardiac electrical signals, deriving a T-wave loop in at least two dimensions, determining a repolarization measurement representative of the T-wave loop; and determining a change in the repolarization measurement from a previously determined repolarization measurement. The method may further include determining a metric of the determined changes in the repolarization measurements and determining when the metric is greater than a risk threshold associated with a cardiac event. The method may further include transmitting a risk notification in response to the metric being greater than the risk threshold.

In yet another example, the disclosure provides a non-transitory computer readable medium storing a set of instructions that, when executed by a control circuit of a medical device, cause the medical device to receive up to two cardiac electrical signals. The instructions further cause the medical device to, for each of multiple cardiac cycles of the received cardiac electrical signal(s) consisting of the up to the two cardiac electrical signals, derive T-wave loop in at least two dimensions, determine a repolarization measurement representative of the T-wave loop; and determine a change in the repolarization measurement from a previously determined repolarization measurement. The instructions may further cause the medical device to determine a metric of the determined changes in the repolarization measurements and determine when the metric is greater than a risk threshold associated with a cardiac event. The instructions may cause the medical device to transmit a risk notification in response to the metric being greater than the risk threshold.

Further disclosed herein is the subject matter of the following examples:

### Example 1

A medical device comprising processing circuitry configured to receive up to two cardiac electrical signals and, for each of a plurality of cardiac cycles of the received cardiac electrical signal(s) consisting of the up to the two cardiac electrical signals, derive a T-wave loop in at least two dimensions. The processing circuitry may determine a repolarization measurement representative of the T-wave loop and determine a change in the repolarization measurement from a previously determined repolarization measurement. The processing circuitry may determine a metric of the determined changes in the repolarization measurements and determine that the metric meets a risk threshold associated with a cardiac event. The medical device may include a telemetry circuit configured to transmit a risk notification in response to the metric meeting the risk threshold.

### Example 2

The medical device of example 1 wherein the processing circuitry is further configured to derive the T-wave loop in at least two dimensions from a first cardiac electrical signal of the up to two cardiac electrical signals by determining a first coordinate in a first dimension and a second coordinate in a second dimension of each point of a plurality of points of the T-wave loop by: determining the first coordinate as a first amplitude of a first sample point of the first cardiac electrical signal, and determining the second coordinate as a second amplitude of a second sample point of the first cardiac electrical signal, the second sample point offset by a first time interval from the first sample point.

### Example 3

The medical device of example 2 wherein the processing circuitry is further configured to derive the T-wave loop in three dimensions from the first cardiac electrical signal by determining a third coordinate in a third dimension of each point of the plurality of points of the T-wave loop as a third amplitude of a third sample point of the first cardiac electrical signal, the third sample point offset by a second time interval from the first sample point.

### Example 4

The medical device of example 3 wherein the processing circuitry is further configured to determine the third amplitude of the third sample point offset by the second time interval from the first sample point where the second time interval is different than the first time interval.

### Example 5

The medical device of example 2 wherein the processing circuitry is further configured to derive the T-wave loop in three dimensions from the first cardiac electrical signal and a second cardiac electrical signal of the up to two cardiac electrical signals by determining a third coordinate in a third dimension of each point of the plurality of points of the T-wave loop as a third amplitude of a third sample point of the second cardiac electrical signal.

### Example 6

The medical device of example 5 wherein the processing circuitry is further configured to identify the third sample point of the second cardiac electrical signal at a common sample time as one of the first sample point of the first cardiac electrical signal or the second sample point of the first cardiac electrical signal.

### Example 7

The medical device of example 1 wherein the processing circuit is further configured to derive the T-wave loop in at least two dimensions from a first cardiac electrical signal and a second cardiac electrical signal of the up to two cardiac electrical signals by determining a first coordinate in a first dimension and a second coordinate in a second dimension of each point of a plurality of points of the T-wave loop by determining the first coordinate as a first amplitude of a first sample point of the first cardiac electrical signal and determining a second coordinate as a second amplitude of a second sample point of the second cardiac electrical signal.

### Example 8

The medical device of example 7 wherein the processing circuitry is further configured to derive the T-wave loop in three dimensions from the first cardiac electrical signal and the second cardiac electrical signal of the up to two cardiac electrical signals by determining a third coordinate of each point of the plurality of points of the T-wave loop by determining a third amplitude from a combination of the first amplitude and the second amplitude.

### Example 9

The medical device of any of examples 1-8 wherein the processing circuitry is further configured to determine the repolarization measurement by determining a T-wave vector in the at least two dimensions from the T-wave loop.

### Example 10

The medical device of example 9 wherein the processing circuity is further configured to determine the change in the repolarization measurement by determining an angle between the T-wave vector and a previously determined T-wave vector.

### Example 11

The medical device of example 9 wherein the processing circuity is further configured to determine an angle between the T-wave vector and an axis of a coordinate system corresponding to the at least two dimensions of the T-wave loop and determine the change in the repolarization measurement by determining a difference between the angle and a previously determined angle between a previously determined T-wave vector and the axis of the coordinate system.

### Example 12

The medical device of any of examples 1-8 wherein the processing circuitry is further configured to determine the repolarization measurement by determining at least one of: an area of the T-wave loop; an area of a two-dimensional projection of the T-wave loop; a distance from a first point of the T-wave loop to a second point of the T-wave loop; a distance from an origin of a coordinate system corresponding to the at least two dimensions of the T-wave loop to a furthest point of the T-wave loop; a centroid of the T-wave loop; or a length of a perimeter of the T-wave loop.

### Example 13

The medical device of any of examples 1-12 wherein the processing circuitry is further configured to determine the metric by a spectral analysis of frequencies of the changes in the repolarization measurements over time.

### Example 14

The medical device of any of examples 1-12 wherein the processing circuitry is further configured to determine the metric by an amplitude analysis of the changes in the repolarization measurement over time.

### Example 15

The medical device of any of examples 1-14 further comprising a therapy delivery circuit configured to deliver or adjust a cardiac electrical stimulation therapy in response to the metric meeting the risk threshold.

### Example 16

The medical device of any of examples 1-15 wherein the processing circuitry is further configured to receive a first cardiac electrical signal of the up to two cardiac electrical signals from a first sensing electrode vector in a horizontal plane of a patient.

### Example 17

The medical device of example 17 wherein the processing circuitry is further configured to receive a second cardiac electrical signal of the up to two cardiac electrical signal from a second sensing electrode vector that is orthogonal to the first sensing electrode vector.

### Example 18

A method performed by a medical device, the method comprising: receiving up to two cardiac electrical signals by processing circuitry of the medical device and, for each of a plurality of cardiac cycles of the received cardiac electrical signal(s) consisting of the up to the two cardiac electrical signals, deriving a T-wave loop in at least two dimensions, determining a repolarization measurement representative of the T-wave loop and determining a change in the repolarization measurement from a previously determined repolarization measurement. The method further includes determining a metric of the determined changes in the repolarization measurements, determining that the metric meets a risk threshold associated with a cardiac event and transmitting a risk notification in response to the metric meeting the risk threshold.

### Example 19

The method of example 18 further comprising deriving the T-wave loop in at least two dimensions from a first cardiac electrical signal of the up to two cardiac electrical signals by determining a first coordinate in a first dimension and a second coordinate in a second dimension of each point of a plurality of points of the T-wave loop by determining the first coordinate as a first amplitude of a first sample point of the first cardiac electrical signal and determining the second coordinate as a second amplitude of a second sample point of the first cardiac electrical signal, the second sample point offset by a first time interval from the first sample point.

### Example 20

The method of example 19, further comprising deriving the T-wave loop in three dimensions from the first cardiac electrical signal by determining a third coordinate in a third dimension of each point of the plurality of points of the T-wave loop as a third amplitude of a third sample point of the first cardiac electrical signal, the third sample point offset by a second time interval from the first sample point.

### Example 21

The method of example 20 further comprising determining the third amplitude of the third sample point offset by the second time interval from the first sample point where the second time interval is different than the first time interval

### Example 22

The method of example 19, further comprising deriving the T-wave loop in three dimensions from the first cardiac electrical signal and a second cardiac electrical signal of the up to two cardiac electrical signals by determining a third coordinate in a third dimension of each point of the plurality of points of the T-wave loop as a third amplitude of a third sample point of the second cardiac electrical signal.

### Example 23

The method of example 22, further comprising identifying the third sample point of the second cardiac electrical signal at a common sample time as one of the first sample point of the first cardiac electrical signal or the second sample point of the first cardiac electrical signal.

### Example 24

The method of example 18, further comprising deriving the T-wave loop in at least two dimensions from a first cardiac electrical signal and a second cardiac electrical signal of the up to two cardiac electrical signals by determining a first coordinate in a first dimension and a second coordinate in a second dimension of each point of a plurality of points of the T-wave loop by determining the first coordinate as a first amplitude of a first sample point of the first cardiac electrical signal and determining a second coordinate as a second amplitude of a second sample point of the second cardiac electrical signal.

### Example 25

The method of example 24, wherein the processing circuitry is further configured to derive the T-wave loop in three dimensions from the first cardiac electrical signal and the second cardiac electrical signal of the up to two cardiac electrical signals by determining a third coordinate of each point of the plurality of points of the T-wave loop by determining a third amplitude from a combination of the first amplitude and the second amplitude.

### Example 26

The method of any of examples 18-25 further comprising determining the repolarization measurement by determining a T-wave vector in the at least two dimensions from the T-wave loop.

### Example 27

The method of example 26 further comprising determining the change in the repolarization measurement by determining an angle between the T-wave vector and a previously determined T-wave vector.

### Example 28

The method of example 26 further comprising determining an angle between the T-wave vector and an axis of a coordinate system corresponding to the at least two dimensions of the T-wave loop and determining the change in the repolarization measurement by determining a difference between the angle and a previously determined angle between a previously determined T-wave vector and the axis of the coordinate system.

### Example 29

The method of any of examples 18-25 further comprising determining the repolarization measurement by determining at least one of: an area of the T-wave loop; an area of a two-dimensional projection of the T-wave loop; a distance from a first point of the T-wave loop to a second point of the T-wave loop; a distance from an origin of a coordinate system corresponding to the at least two dimensions of the T-wave loop to a furthest point of the T-wave loop; a centroid of the T-wave loop; or a length of a perimeter of the T-wave loop.

### Example 30

The method of any of examples 18-29 further comprising determining the metric by a spectral analysis of frequencies of the changes in the repolarization measurements over time.

### Example 31

The method of any of examples 18-29, further comprising determining the metric by an amplitude analysis of the changes in the repolarization measurement over time.

### Example 32

The method of any of examples 18-31 further comprising delivering or adjusting a cardiac electrical stimulation therapy in response to the metric meeting the risk threshold.

### Example 33

The method of any of examples 18-32 further comprising receiving a first cardiac electrical signal of the up to two cardiac electrical signals from a first sensing electrode vector in a horizontal plane of a patient.

### Example 34

The method of example 33 further comprising receiving a second cardiac electrical signal of the up to two cardiac electrical signal from a second sensing electrode vector that is orthogonal to the first sensing electrode vector.

### Example 35

A non-transitory, computer readable medium storing a set of instructions that, when executed by a control circuit of a medical device, cause the medical device to: receive up to two cardiac electrical signals and, for each of a plurality of cardiac cycles of the received cardiac electrical signal(s) consisting of the up to the two cardiac electrical signals, derive a T-wave loop in at least two dimensions, determine a repolarization measurement representative of the T-wave loop and determine a change in the repolarization measurement from a previously determined repolarization measurement. The instructions may further cause the medical device to determine a metric of the determined changes in the repolarization measurements, determine that the metric meets a risk threshold associated with a cardiac event and transmit a risk notification in response to the metric meeting the risk threshold.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIGS. 1A and 1B are conceptual diagrams of one example of a medical device system that may be configured to sense cardiac electrical signals and determine a metric of changes in repolarization of the myocardium for assessing the risk of a cardiac event according to the techniques disclosed herein.

FIGS. 2A-2C are conceptual diagrams of a patient implanted with a medical device system in a different implant configuration than the arrangement shown in FIGS. 1A-1B.

FIG. 3 is a conceptual diagram of another example of a medical device system that may be configured to perform the techniques disclosed herein.

FIG. 4 is a conceptual diagram of one example of a leadless medical device that may be configured to sense at least one cardiac electrical signal and determine a metric of changes in repolarization according to the techniques disclosed herein.

FIGS. 5A and 5B are conceptual diagrams of other examples of leadless medical devices that may be configured to sense at least one cardiac electrical signal and determine a metric of changes in repolarization according to the techniques disclosed herein.

FIG. 6 is a conceptual diagram of a medical device configured to perform the techniques disclosed herein according to some examples.

FIG. 7 is a flow chart of a method performed by a medical device for determining a metric of cardiac repolarization changes for predicting risk of a cardiac event, such as sudden cardiac death.

FIG. 8 is a flow chart of a method for deriving repolarization measurements from a single cardiac electrical signal received by processing circuitry of a medical or computing device.

FIG. 9A is a diagram of a T-wave that may be sensed during a T-wave window.

FIG. 9B is a diagram of a T-wave loop that may be generated by processing circuitry from a single cardiac electrical signal.

FIG. 10 is an illustrative plot of the of determined changes in repolarization measurements (A RM) that may be accumulated in memory of a medical device over a specified time period or number of cardiac cycles.

FIG. 11 is diagram of an example 3D T-wave loop that may be generated from a single cardiac electrical signal.

FIG. 12 is a diagram of two T-wave vectors, each representative of a T-wave loop determined from a single cardiac cycle, that may be determined by processing circuitry of a medical device according to some examples.

FIG. 13 is a flow chart of a method for determining a metric of repolarization changes for predicting risk of a cardiac event according to another example.

FIG. 14 is a diagram of two cardiac electrical signals and that may be received by medical device processing circuitry for use in determining T-wave loops and a metric of repolarization changes.

### DETAILED DESCRIPTION

In general, this disclosure describes a medical device and techniques for determining a metric indicative of a patient's risk of a serious cardiac event, such as tachyarrhythmia or sudden cardiac death. In various examples, the medical device performing the techniques disclosed herein includes processing circuitry for receiving up to two cardiac electrical signals and determining repolarization measurements from the T-waves of multiple cardiac cycles of one received cardiac electrical signal or two received cardiac electrical signals. In various examples described herein, the repolarization measurements may be determined by the processing circuitry by deriving a two dimensional (2D) or three dimensional (3D) T-wave loop from the cardiac electrical signal(s) received during a T-wave window and determining the repolarization measurement representative of the T-wave loop. Changes in the repolarization measurements may be quantified by determining a metric from the changes over time that is indicative of a patient's risk of a serious cardiac event.

The medical device and techniques disclosed herein provide various improvements in a medical device configured to predict a cardiac event or identify patients at risk of experiencing a serious cardiac event to enable early or prophylactic treatment for preventing or reducing the severity of the event. The techniques disclosed herein improve the function of a medical device in providing an indication of risk of a cardiac event by reducing the number of cardiac electrical signals required to determine a metric of changes in repolarization of the myocardium that is indicative of the risk of a cardiac event. By reducing the number of cardiac electrical signals required to determine the metric, processing time and power required to determine the metric may be reduced, allowing the techniques for assessing patient risk to be implemented in a variety of medical or computing devices configured to sense or receive at least one cardiac electrical signal.

The techniques disclosed herein therefore provide improvements in the computer-related field of cardiac monitoring and cardiac therapy delivery. By providing a medical device system capable of determining a metric of changes in repolarization according to the techniques herein, the complexity and likelihood of human error in identifying patients that could benefit from various treatments, e.g., pharmacological and/or implantable medical devices such as pacemakers or implantable cardioverter defibrillation, can be reduced. Lifesaving treatments can be provided for patients that can be identified as having a risk of a cardiac event. The techniques disclosed herein can reduce the time burden and expertise required of a clinician in interpreting cardiac electrical signals for identifying a patient at risk of a serious cardiac event. The techniques disclosed herein may enable a risk notification to be transmitted or displayed by the medical device and/or a therapy to be delivered for reducing the likelihood or preventing the cardiac event when the risk is identified with a relatively a high degree of confidence in a manner that is simplified, flexible, and patient specific.

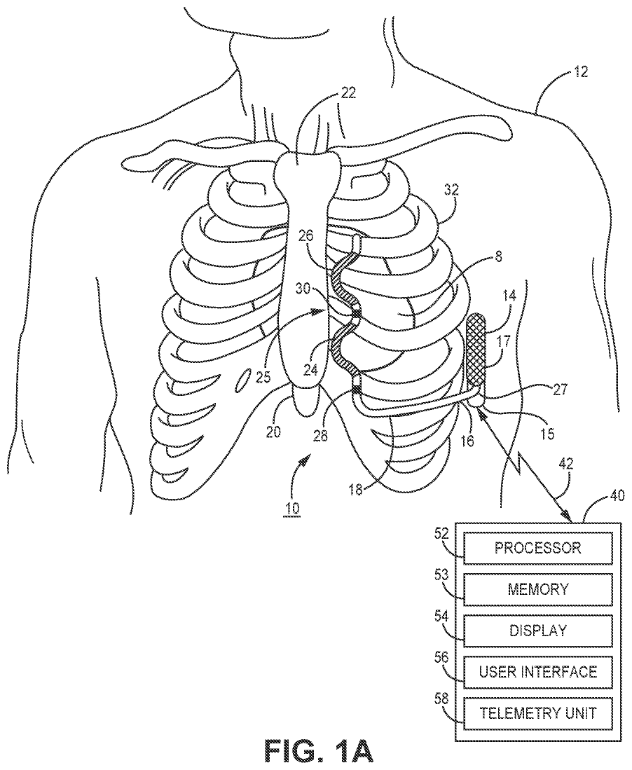

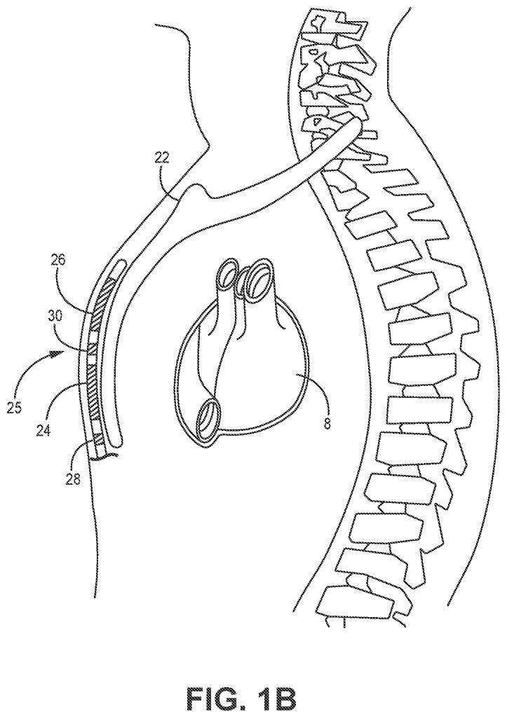

FIGS. 1A and 1B are conceptual diagrams of one example of a medical device system 10 that may be configured to sense cardiac electrical signals and determine a metric of changes in repolarization of the myocardium for assessing the risk of a cardiac event according to the techniques disclosed herein. FIG. 1A is a front view of the medical system 10 implanted within patient 12. FIG. 1B is a side view of the medical device system 10 implanted within patient 12. Medical device system 10 includes an implantable medical device (IMD) 14 connected to at least one medical lead 16. Medical lead 16 can be used for sensing at least one cardiac electrical signal and may be used for delivering electrical stimulation therapies when IMD 14 is capable of delivering electrical stimulation therapies, such as cardiac pacing, CV/DF shocks, or neurostimulation therapy. For the sake of illustration, FIGS. 1A and 1B are described in the context of IMD 14 being an implantable cardioverter defibrillator (ICD) capable of providing high voltage CV/DF shocks and/or cardiac pacing pulses in response to detecting a cardiac arrhythmia based on processing of sensed cardiac electrical signals.

The techniques for determining a metric of changes in repolarization of the myocardium as disclosed herein, however, may be implemented in a cardiac monitoring device that does not necessarily include therapy delivery capabilities. In other examples, the techniques disclosed herein may be implemented in a device capable of delivering one or more therapies other than cardiac electrical stimulation therapies, such a neurostimulation therapy and/or drug delivery. For example, the techniques disclosed herein may be implemented in a medical device configured to deliver neurostimulation to the vagal nerve or another nervous system site for altering autonomic tone. The techniques disclosed herein may be implemented in a medical device that includes a drug pump configured to deliver a pharmacological agent that may reduce the likelihood of myocardial infarct, reduce the likelihood of arrhythmia, or otherwise reduce the likelihood of a serious or life-threatening cardiac event. The techniques disclosed herein for sensing at least one cardiac electrical signal and determining metric indicative of a risk of a cardiac event may be implemented in a variety of medical devices including external or implantable medical devices or computing devices, including handheld or wearable devices such as a fitness tracker, tablet, smart phone, or other device.

IMD 14 includes a housing 15 that forms a hermetic seal that protects internal components of IMD 14. The housing 15 of IMD 14 may be formed of a conductive material, such as titanium or titanium alloy. The housing 15 may function as an electrode (sometimes referred to as a “can” electrode). Housing 15 may be used as an active can electrode for use in delivering CV/DF shocks or other high voltage pulses delivered using a high voltage therapy circuit. In other examples, housing 15 may be available for use in delivering unipolar, relatively lower voltage cardiac pacing pulses and/or for sensing cardiac electrical signals in combination with electrodes carried by lead 16. In other instances, the housing 15 of IMD 14 may include a plurality of electrodes on an outer portion of the housing. The outer portion(s) of the housing 15 functioning as an electrode(s) may be coated with a material, such as titanium nitride, e.g., for reducing post-stimulation polarization artifact.

IMD 14 includes a connector assembly 17 (also referred to as a connector block or header) that includes electrical feedthroughs crossing housing 15 to provide electrical connections between conductors extending within the lead body 18 of lead 16 and electronic components included within the housing 15 of IMD 14. As will be described in further detail herein, housing 15 may house one or more processing circuits for analyzing cardiac signals and controlling IMD functions, memories, transceivers, cardiac electrical signal sensing circuitry, therapy delivery circuitry, power source(s) and/or other components for sensing cardiac electrical signals, detecting a heart rhythm, and controlling and delivering electrical stimulation pulses to treat an abnormal heart rhythm and/or to reduce the likelihood of a serious cardiac event that is predicted based on a metric of changes in repolarization determined according to the techniques disclosed herein.

Lead 16 includes an elongated lead body 18 having a proximal end 27 that includes a lead connector (not shown) configured to be connected to IMD connector assembly 17 and a distal portion 25 that includes one or more electrodes. In the example illustrated in FIGS. 1A and 1B, the distal portion 25 of lead body 18 includes defibrillation electrodes 24 and 26 and pace/sense electrodes 28 and 30. In some cases, defibrillation electrodes 24 and 26 may together form a defibrillation electrode in that they may be configured to be activated concurrently. Alternatively, defibrillation electrodes 24 and 26 may form separate defibrillation electrodes in which case each of the electrodes 24 and 26 may be activated independently.

Electrodes 24 and 26 (and in some examples housing 15) are referred to herein as defibrillation electrodes because they are utilized, individually or collectively, for delivering high voltage stimulation therapy (e.g., CV/DF shocks) for terminating a tachyarrhythmia. Electrodes 24 and 26 may be elongated coil electrodes and generally have a relatively high surface area for delivering high voltage electrical stimulation pulses compared to pacing and sensing electrodes 28 and 30. However, electrodes 24 and 26 and housing 15 may also be utilized to provide pacing functionality, sensing functionality or both pacing and sensing functionality in addition to or instead of high voltage stimulation therapy. In this sense, the use of the term “defibrillation electrode” herein should not be considered as limiting the electrodes 24 and 26 for use in only high voltage CV/DF shock therapy delivery. For example, either or both of electrodes 24 and 26 may be used as a sensing electrode in a sensing electrode vector for sensing at least one cardiac electrical signal used for determining a metric of repolarization changes used in assessing a patient's risk of a future cardiac event.

Electrodes 28 and 30 are relatively smaller surface area electrodes which are available for use in sensing electrode vectors for sensing cardiac electrical signals and may be used for delivering relatively low voltage pacing pulses in some configurations. Electrodes 28 and 30 are referred to as pace/sense electrodes because they are generally configured for use in low voltage applications, e.g., used as either a cathode or anode for delivery of pacing pulses and/or sensing of cardiac electrical signals, as opposed to delivering high voltage CV/DF shocks. In some instances, electrodes 28 and 30 may provide only pacing functionality, only sensing functionality or both.

IMD 14 may obtain cardiac electrical signals corresponding to electrical activity of heart 8 via a combination of sensing electrode vectors that include combinations of electrodes 24, 26, 28 and/or 30. In some examples, housing 15 of IMD 14 is used in combination with one or more of electrodes 24, 26, 28 and/or 30 in at least one sensing electrode vector. Various sensing electrode vectors utilizing combinations of electrodes 24, 26, 28, and 30 and housing 15 are described below for sensing one or more cardiac electrical signals that may be used in acquiring up to two cardiac electrical signals that may be used in determining a metric of changes in repolarization of the myocardium. Each cardiac electrical signal that is sensed by IMD 14 may be sensed using a different sensing electrode vector, which may be selected by sensing circuitry included in IMD 14. In some examples the cardiac electrical signal(s) received via a selected sensing electrode vector may be used by IMD 14 for sensing R-waves attendant to ventricular depolarization and/or P-waves attendant to atrial depolarization. R-waves and P-waves may be referred to herein as “depolarization signals” or “cardiac depolarization signals.” Sensed R-waves and/or P-waves may be used by IMD processing circuitry for determining the heart rate and determining a need for cardiac pacing, e.g., for treating bradycardia or asystole for preventing a long ventricular pause, or for determining a need for tachyarrhythmia therapies, e.g., anti-tachycardia pacing (ATP) or CV/DF shocks.

At least one cardiac electrical signal may be sensed by IMD 14 using a sensing electrode vector selected from the available electrodes 24, 26, 28, 30 and housing 15 for obtaining T-wave signals attendant to myocardial repolarizations. The T-wave signals, which may also be referred to herein as “repolarization signals,” may be used by processing circuitry of IMD 14 for determining a repolarization measurement from each of a multiple cardiac cycles. As described in greater detail below, changes in the repolarization measurements determined from successive T-waves of one or up to two cardiac electrical may be quantified for determining a patient's risk of having a serious or life-threatening cardiac event, such as sudden cardiac death.

In the example illustrated in FIGS. 1A and 1B, electrode 28 is located proximal to defibrillation electrode 24, and electrode 30 is located between defibrillation electrodes 24 and 26. One, two or more pace/sense electrodes may be carried by lead body 18. For instance, a third pace/sense electrode may be located distal to defibrillation electrode 26 in some examples. Electrodes 28 and 30 are illustrated as ring electrodes; however, electrodes 28 and 30 may comprise any of a number of different types of electrodes, including ring electrodes, short coil electrodes, hemispherical electrodes, directional electrodes, segmented electrodes, or the like. Electrodes 28 and 30 may be positioned at other locations along lead body 18 and are not limited to the positions shown. In other examples, lead 16 may include fewer or more pace/sense electrodes and/or defibrillation electrodes than the example shown here.

In the example shown, lead 16 is a non-transvenous lead that may extend subcutaneously or submuscularly over the ribcage 32 medially from the connector assembly 27 of IMD 14 toward a center of the torso of patient 12, e.g., toward xiphoid process 20 of patient 12. At a location near xiphoid process 20, lead 16 bends or turns and extends superiorly, subcutaneously or submuscularly, over the ribcage and/or sternum, substantially parallel to sternum 22. Although illustrated in FIG. 1A as being offset laterally from and extending substantially parallel to sternum 22, the distal portion 25 of lead 16 may be implanted at other locations, such as over sternum 22, offset to the right or left of sternum 22, angled laterally from sternum 22 toward the left or the right, or the like. Alternatively, lead 16 may be placed along other subcutaneous or submuscular paths. The path of lead 16 may depend on the location of IMD 14, the arrangement and position of electrodes carried by the lead body 18, and/or other factors. The techniques disclosed herein are not necessarily limited to a particular path of lead 16 or final locations of electrodes 24, 26, 28 and 30. It is recognized, however, that some sensing electrode vectors used for sensing up to two cardiac electrical signals used in assessing a patient's risk for a cardiac event may provide greater confidence in predicting a cardiac event than other sensing electrode vectors. For example, the T-wave associated with myocardial repolarization may have a greater signal strength along some sensing electrode vectors compared to other sensing electrode vectors and/or periodic changes in the T-wave may be more pronounced along some sensing electrode vectors than other sensing electrode vectors.

Electrical conductors (not illustrated) extend through one or more lumens of the elongated lead body 18 of lead 16 from the lead connector at the proximal lead end 27 to electrodes 24, 26, 28, and 30 located along the distal portion 25 of the lead body 18. The elongated electrical conductors contained within the lead body 18, which may be separate respective insulated conductors within the lead body 18, are each electrically coupled with respective defibrillation electrodes 24 and 26 and pace/sense electrodes 28 and 30. The respective conductors electrically couple the electrodes 24, 26, 28, and 30 to circuitry, such as a therapy delivery circuit and/or a sensing circuit, of IMD 14 via connections in the connector assembly 17, including associated electrical feedthroughs crossing housing 15. The electrical conductors transmit electrical stimulation pulses from a therapy delivery circuit within IMD 14 to one or more of defibrillation electrodes 24 and 26 and/or pace/sense electrodes 28 and 30 and transmit electrical signals produced by the patient's heart 8 from one or more of defibrillation electrodes 24 and 26 and/or pace/sense electrodes 28 and 30 to the sensing circuitry within IMD 14.

The lead body 18 of lead 16 may be formed from a non-conductive material, including silicone, polyurethane, fluoropolymers, mixtures thereof, and/or other appropriate materials, and shaped to form one or more lumens within which the one or more conductors extend. Lead body 18 may be tubular or cylindrical in shape. In other examples, the distal portion 25 (or all of) the elongated lead body 18 may have a flat, ribbon or paddle shape. Lead body 18 may be formed having a preformed distal portion 25 that is generally straight, curving, bending, serpentine, undulating or zig-zagging.

In the example shown, lead body 18 includes a curving distal portion 25 having two “C” shaped curves, which together may resemble the Greek letter epsilon, “E.” Defibrillation electrodes 24 and 26 are each carried by one of the two respective C-shaped portions of the lead body distal portion 25. The two C-shaped curves are seen to extend or curve in the same direction away from a central axis of lead body 18, along which pace/sense electrodes 28 and 30 are positioned. Pace/sense electrodes 28 and 30 may, in some instances, be approximately aligned with the central axis of the straight, proximal portion of lead body 18 such that mid-points of defibrillation electrodes 24 and 26 are laterally offset from pace/sense electrodes 28 and 30.

Other examples of extra-cardiovascular leads may include one or more defibrillation electrodes and/or one or more pacing and sensing electrodes carried by a curving, serpentine, undulating or zig-zagging distal portion of the lead body 18. The techniques disclosed herein are not limited to any particular lead body design, however. In other examples, lead body 18 is a flexible elongated lead body without any pre-formed shape, bends or curves.

IMD 14 may be configured to analyze the cardiac electrical signal(s) received from one or more sensing electrode vectors to monitor for abnormal rhythms, such as asystole, bradycardia, ventricular tachycardia (VT) and/or ventricular fibrillation (VF). IMD 14 may analyze the heart rate and/or morphology of the cardiac electrical signals to monitor for tachyarrhythmia in accordance with tachyarrhythmia detection techniques. IMD 14 may generate and deliver electrical stimulation therapy in response to detecting a tachyarrhythmia, e.g., VT or VF (VT/VF) using a therapy delivery electrode vector which may be selected from any of the available electrodes 24, 26, 28 30 and/or housing 15. IMD 14 may deliver ATP in response to VT detection and in some cases may deliver ATP prior to a CV/DF shock or during high voltage capacitor charging in an attempt to avert the need for delivering a CV/DF shock. If ATP does not successfully terminate VT or when VF is detected, IMD 14 may deliver one or more CV/DF shocks via one or both of defibrillation electrodes 24 and 26 and/or housing 15.

In the absence of a sensed R-wave, IMD 14 may generate and deliver a cardiac pacing pulse, such as a post-shock pacing pulse or bradycardia pacing pulse when asystole is detected or when a pacing escape interval expires prior to sensing a ventricular event signal, e.g., when AV block is present. The cardiac pacing pulses may be delivered using a pacing electrode vector that includes one or more of the electrodes 24, 26, 28, and 30 and the housing 15 of IMD 14.

As described below, at least one sensing electrode vector may be selected for sensing a cardiac electrical signal during multiple T-wave windows. The cardiac electrical signal sensed during T-wave windows of multiple cardiac cycles may be received by processing circuitry of IMD 14 and analyzed for determining a metric of changes in repolarization that can be compared to a risk threshold. Electrodes 24, 26, 28, 30 and/or housing 15 may be selected in one or more therapy delivery electrode vectors for delivering an electrical stimulation therapy to reduce the likelihood of a cardiac event associated with the risk threshold when the metric meets, e.g., exceeds, the risk threshold.

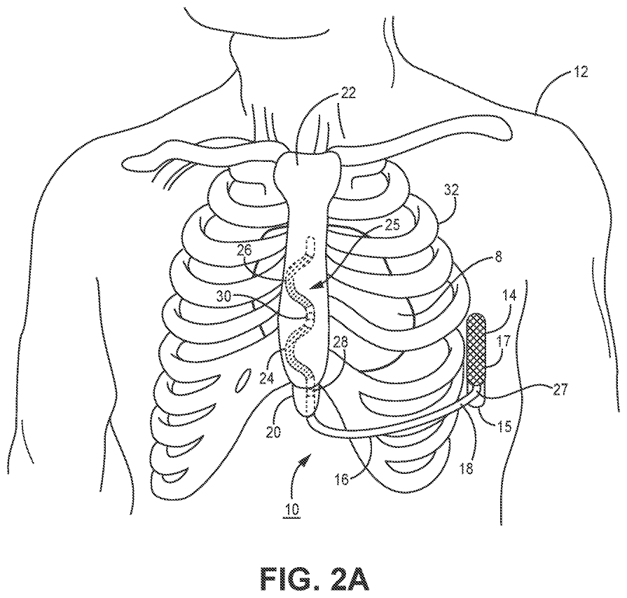

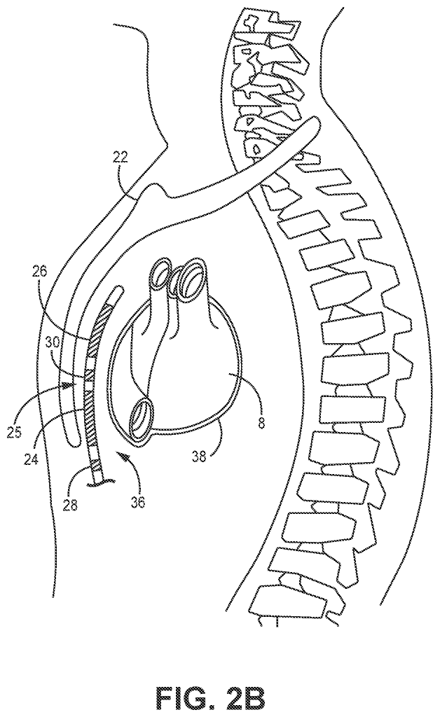

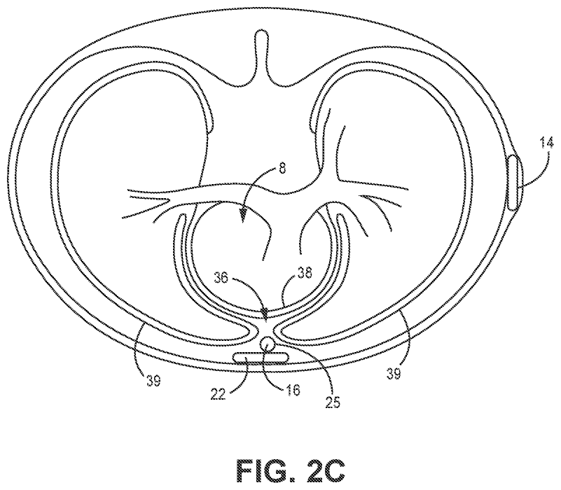

IMD 14 is shown implanted subcutaneously on the left side of patient 12 along the ribcage 32. IMD 14 may, in some instances, be implanted between the left posterior axillary line and the left anterior axillary line of patient 12. IMD 14 may, however, be implanted at other subcutaneous or submuscular locations in patient 12. For example, IMD 14 may be implanted in a subcutaneous pocket in the pectoral region. In this case, lead 16 may extend subcutaneously or submuscularly from IMD 14 toward the manubrium of sternum 22 and bend or turn and extend inferiorly from the manubrium to the desired location subcutaneously or submuscularly. In yet another example, IMD 14 may be placed abdominally. Lead 16 may be implanted in other extra-cardiovascular locations as well. For instance, as described with respect to FIGS. 2A-2C, the distal portion 25 of lead 16 may be implanted underneath the sternum/ribcage in the substernal space. FIGS. 1A and 1B are illustrative in nature and should not be considered limiting in the practice of the techniques disclosed herein.

A medical device operating according to techniques disclosed herein may be coupled to a transvenous or non-transvenous lead in various examples for carrying electrodes for sensing cardiac electrical signals and, in some examples, delivering electrical stimulation therapy. For example, the medical device, such as IMD 14, may be coupled to an extra-cardiovascular lead as illustrated in the accompanying drawings, referring to a lead that positions electrodes outside the blood vessels, heart, and pericardium surrounding the heart of a patient. Implantable electrodes carried by extra-cardiovascular leads may be positioned extra-thoracically (outside the ribcage and sternum), subcutaneously or submuscularly, or intra-thoracically (beneath the ribcage or sternum, sometimes referred to as a sub-sternal position) and may not necessarily be in intimate contact with myocardial tissue. An extra-cardiovascular lead may also be referred to as a “non-transvenous”lead.

In other examples, the medical device may be coupled to a transvenous lead that positions electrodes within a blood vessel, which may remain outside the heart in an “extra-cardiac” location or be advanced to position electrodes within a heart chamber. For instance, a transvenous medical lead may be advanced along a venous pathway to position electrodes in an extra-cardiac location within the internal thoracic vein (ITV), an intercostal vein, the superior epigastric vein, or the azygos, hemiazygos, or accessory hemiazygos veins, as examples. In still other examples, a transvenous lead may be advanced to position electrodes within the heart, e.g., within an atrial and/or ventricular heart chamber or within a cardiac vein.

An external device 40 is shown in telemetric communication with IMD 14 by a wireless communication link 42 in FIG. 1A. External device 40 may include a processor 52, memory 53, display 54, user interface 56 and telemetry unit 58. Processor 52 controls external device operations and processes data and signals received from IMD 14. Display unit 54, which may include a graphical user interface, displays data and other information to a user for reviewing IMD operation and programmed parameters as well as cardiac electrical signals retrieved from IMD 14.

User interface 56 may include a mouse, touch screen, keypad or the like to enable a user to interact with external device 40 to initiate a telemetry session with IMD 14 for retrieving data from and/or transmitting data to IMD 14, including programmable parameters for controlling cardiac event signal sensing, arrhythmia detection and therapy delivery. Telemetry unit 58 includes a transceiver and antenna configured for bidirectional communication with a telemetry circuit included in IMD 14 and is configured to operate in conjunction with processor 52 for sending and receiving data relating to IMD functions via communication link 42.

Communication link 42 may be established between IMD 14 and external device 40 using a radio frequency (RF) link such as BLUETOOTH®, Wi-Fi, or Medical Implant Communication Service (MICS) or other RF or communication frequency bandwidth or communication protocols. Data stored or acquired by IMD 14, including physiological signals or associated data derived therefrom, results of device diagnostics, battery status, and histories of detected rhythm episodes and delivered therapies, etc., may be retrieved from IMD 14 by external device 40 following an interrogation command.

External device 40 may be embodied as a programmer used in a hospital, clinic or physician's office to retrieve data from IMD 14 and to program operating parameters and algorithms in IMD 14 for controlling ICD functions. External device 40 may alternatively be embodied as a home monitor or handheld device. External device 40 may be used to program cardiac signal sensing parameters, cardiac rhythm detection parameters and therapy control parameters used by IMD 14. At least some control parameters used in sensing cardiac event signals and detecting arrhythmias according to the techniques disclosed herein as well as therapy delivery may be programmed into IMD 14 using external device 40 in some examples.

As described herein, IMD 14 may transmit a notification in response to determining that a metric of changes in repolarization measurements determined from T-waves of one cardiac electrical signal or up to two cardiac electrical signals meets a risk threshold. Display unit 54 may display an alert or an alarm in response to external device 40 receiving the notification. External device 40 may be used to program the risk threshold and/or other control parameters used for determining the metric of changes in repolarization measurements. Such control parameters may include the sensing electrode vector(s), the number of cardiac electrical signals used for determining the metric, control parameters used in computing the metric, or the like. External device 40 may be used by a clinician to program IMD 14 to respond to a metric meeting the risk threshold by adjusting a therapy. As used herein “adjusting a therapy” may refer to starting a therapy, stopping a therapy, and/or altering a therapy that is being delivered, e.g., by altering a rate, dosage or other therapy control parameter.

FIGS. 2A-2C are conceptual diagrams of patient 12 implanted with medical device system 10 in a different implant configuration than the arrangement shown in FIGS. 1A-1B. FIG. 2A is a front view of patient 12 implanted with medical device system 10. FIG. 2B is a side view of patient 12 implanted with medical device system 10. FIG. 2C is a transverse view of patient 12 implanted with medical device system 10. In this arrangement lead 16 of system 10 is implanted at least partially underneath sternum 22 of patient 12. Lead 16 may extend subcutaneously or submuscularly from IMD 14 toward xiphoid process 20 and at a location near xiphoid process 20 bends or turns and extends superiorly within anterior mediastinum 36 (see FIG. 2C) in a substernal position.

Anterior mediastinum 36 may be viewed as being bounded laterally by pleurae 39, posteriorly by pericardium 38, and anteriorly by sternum 22 (see FIG. 2C). The distal portion 25 of lead 16 may extend along the posterior side of sternum 22 substantially within the loose connective tissue and/or substernal musculature of anterior mediastinum 36. A lead implanted such that the distal portion 25 is substantially within anterior mediastinum 36, may be referred to as a “substernal lead.”

In the example illustrated in FIGS. 2A-2C, lead 16 is located substantially centered under sternum 22. In other instances, however, lead 16 may be implanted such that it is offset laterally from the center of sternum 22. In some instances, lead 16 may extend laterally such that distal portion 25 of lead 16 is underneath/below the ribcage 32 in addition to or instead of sternum 22. In other examples, the distal portion 25 of lead 16 may be implanted in other extra-cardiac, intra-thoracic locations, including in the pleural cavity or around the perimeter of and adjacent to the pericardium 38 of heart 8.

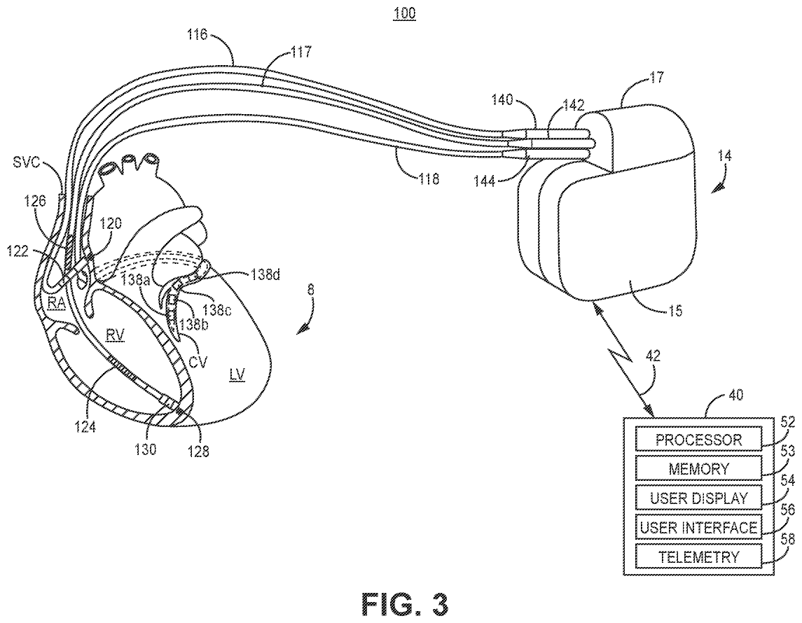

FIG. 3 is a conceptual diagram of another example of a medical device system 100 that may be configured to perform the techniques disclosed herein. Medical device system 100 includes IMD 14 coupled to transvenous leads 116, 117 and 118 for sensing cardiac electrical signals and delivering cardiac electrical stimulation therapy in each of the right atrium (RA), right ventricle (RV) and left ventricle (LV) of heart 8. In this example, IMD 14 may be configured as a multi-chamber pacemaker and defibrillator capable of delivering cardiac resynchronization therapy (CRT). CRT includes delivering pacing pulses in the LV, RV and/or RA for improving mechanical synchrony of the right and left ventricles with each other and/or with the atria, which may promote more efficient pumping of the heart 8. Accordingly, IMD 14 is coupled to three leads 116, 117 and 118 in this example to provide multi-chamber sensing and pacing. IMD 14 may additionally be capable of delivering high voltage cardioversion or defibrillation (CV/DF) shocks for treating cardiac tachyarrhythmias.

In other examples, however, the techniques disclosed herein may be implemented in a single chamber, dual chamber or multi-chamber cardiac pacemaker, with or without CV/DF capabilities. Furthermore, it is to be understood that any IMD capable of sensing a cardiac electrical signal that includes T-wave signals attendant to ventricular myocardial repolarizations may be adapted to perform the techniques disclosed herein. The multi-chamber cardiac sensing and cardiac pacing therapy capabilities described in conjunction with IMD 14 when coupled to multiple transvenous leads are not required for practicing the presently disclosed techniques for monitoring T-wave signals for determining a metric of changes in repolarization measurements indicative of a patient's risk for a cardiac event.

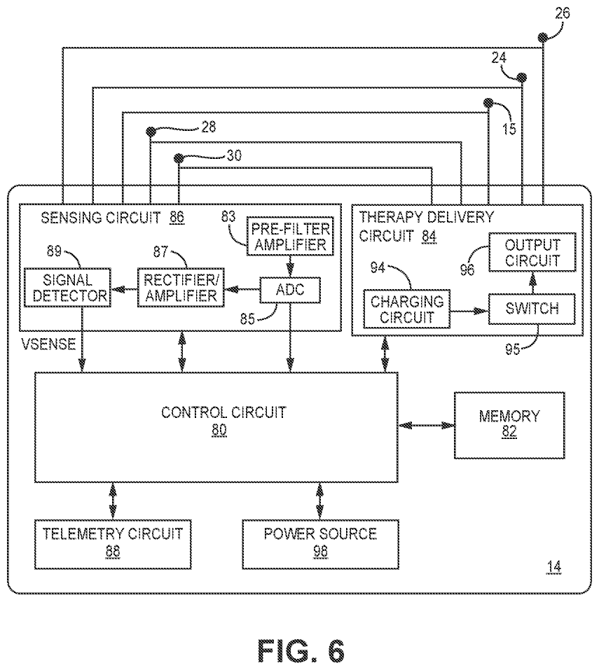

As described above IMD 14 can include a connector assembly 17 coupled to a housing 15 that encloses circuitry configured to perform IMD functions, such as a processor, cardiac electrical signal sensing circuitry and therapy delivery circuitry as further described in conjunction with FIG. 6, below. Connector assembly 17, sometimes referred to as a “header,” is hermetically sealed to housing 15 and includes, in this example, three connector bores for receiving proximal lead connectors 140, 142 and 144 of each of the respective leads 116, 117 and 118 to provide electrical communication between electrodes carried by the distal portion of each lead and the sensing and therapy delivery circuitry enclosed by housing 15.

Leads coupled to IMD 14 may include RA lead 116, RV lead 117 and a coronary sinus (CS) lead 118. RA lead 116 may carry a distal tip electrode 120 and ring electrode 122 spaced proximally from tip electrode 120 for sensing atrial electrical signals, e.g., P-waves, and delivering RA pacing pulses. RA lead 116 may be positioned such that its distal end is in the vicinity of the RA and the superior vena cava and includes insulated electrical conductors extending through the elongated lead body from each of electrodes 120 and 122 to the proximal lead connector 140.

RV lead 117 includes pacing and sensing electrodes 128 and 130 shown as a tip electrode 128 and a ring electrode 130 spaced proximally from tip electrode 128. The electrodes 128 and 130 provide sensing and pacing in the RV and are each connected to a respective insulated conductor within the body of RV lead 117. Each insulated conductor is coupled at its proximal end to proximal lead connector 142. RV lead 117 is positioned such that its distal end is in the RV for sensing RV electrical signals, such as R-waves attendant to ventricular depolarizations and T-waves attendant to ventricular repolarizations and delivering pacing pulses in the RV. In some examples, IMD 14 is capable of delivering high voltage pulses for cardioverting or defibrillating heart 8 in response to detecting a tachyarrhythmia. In this case, RV lead 117 may include defibrillation electrodes 124 and 126, which may be elongated coil electrodes used to deliver high voltage CV/DF therapy, also referred to a “shocks” or “shock pulses.”

Defibrillation electrode 124 may be referred to as the “RV defibrillation electrode” or “RV coil electrode” because it is carried along the body of RV lead 117 such that it is positioned substantially within the RV when distal pacing and sensing electrodes 128 and 130 are positioned for pacing and sensing in the RV. For example, tip electrode 128 may be positioned at an endocardial location of the RV apex or along the interventricular septum. Defibrillation electrode 126 may be referred to as a “superior vena cava (SVC) defibrillation electrode” or “SVC coil electrode” because it is carried along the body of RV lead 117 such that it is positioned at least partially along the SVC when the distal end of RV lead 117 is advanced within the RV. The IMD housing 15 may serve as a subcutaneous defibrillation electrode in combination with one or both of RV coil electrode 124 and SVC coil electrode 126 for delivering CV/DF shocks to heart 8. While electrodes 124 and 126 are referred to herein as defibrillation electrodes, it is to be understood that electrodes 124 and 126 may be used for sensing cardiac electrical signals, delivering cardiac pacing pulses or delivering anti-tachycardia pacing (ATP) therapy and are not necessarily limited to only being used for delivering high voltage CV/DV shock pulses. In some examples, any of electrodes 124, 126, 128 and 130 of RV lead 117 may be used in sensing T-wave signals for deriving T-wave loops and determining a metric indicative of a cardiac event risk according to the techniques disclosed herein. Each of electrodes 124, 126, 128 and 130 are connected to a respective insulated conductor extending within the body of lead 117. The proximal end of the insulated conductors are coupled to corresponding connectors carried by proximal lead connector 142, e.g., a DF-4 connector, at the proximal end of lead 117 for providing electrical connection to IMD 14.

CS lead 118 may be advanced within the vasculature of the left side of the heart via the coronary sinus and a cardiac vein (CV). CS lead 118 may include one or more electrodes for sensing cardiac electrical signals and delivering pacing pulses to the LV. CS lead 118 is shown as a quadripolar lead having four electrodes 138a, 138b, 138c, and 138d, collectively “electrodes 138,” that may be selected in various bipolar or unipolar electrode vectors for sensing cardiac electrical signals from the LV and delivering cardiac pacing pulses to the LV, e.g., during CRT delivery. The electrodes 138 are each coupled to respective insulated conductors within the body of CS lead 118 which provide electrical connection to the proximal lead connector 144, coupled to IMD connector assembly 17.

The various electrodes 120, 122, 124, 126, 128, 130, 138 and housing 15 may be selected in a variety of unipolar and/or bipolar sensing electrode vectors for sensing T-wave signals for determining a metric of changes in repolarization for assessing cardiac event risk in a patient according to the techniques disclosed herein. It is recognized that numerous sensing and electrical stimulation electrode vectors may be available using the various electrodes carried by one or more of leads 116, 117 and 118. Alternate transvenous lead systems may be substituted for the three lead system illustrated in FIG. 3. For example, a medical device performing the techniques disclosed herein may be coupled to one or more transvenous leads, such as leads 116, 117 and 118 and/or one or more extra-cardiac leads that extend subcutaneously, submuscularly or substernally.

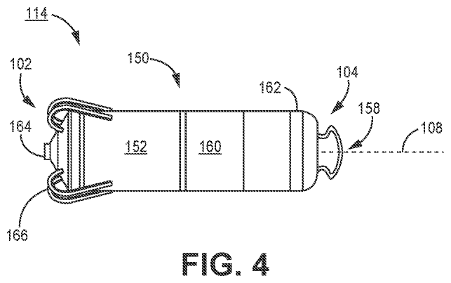

FIG. 4 is a conceptual diagram of one example of a leadless medical device that may be configured to sense at least one cardiac electrical signal and determine a metric of changes in repolarization according to the techniques disclosed herein. In the examples described above in conjunction with FIGS. 1A-3, IMD 14 is shown coupled to a medical electrical lead carrying electrodes for sensing at least one cardiac electrical signal. In other examples, an IMD configured to perform the techniques disclosed herein may be a lead medical device, carrying electrodes on the housing of the IMD. IMD 114 shown in FIG. 4 includes electrodes 162 and 164 spaced apart along the housing 150 of IMD 114 for sensing a cardiac electrical signal. IMD 114 may be configured as a leadless pacemaker configured to sense a cardiac electrical signal and deliver cardiac pacing pulses from electrodes 162 and 164. IMD 114 may be configured to be implanted wholly within a heart chamber, e.g., within an atrial or a ventricular heart chamber. Housing 150 may be generally cylindrical for facilitating delivery by a delivery device, such as a transvenous catheter.

Electrode 164 is shown as a tip electrode extending from a distal end 102 of IMD 114, and electrode 162 is shown as a ring electrode along a mid-portion of housing 150, for example adjacent proximal end 104. Distal end 102 is referred to as “distal” in that it is expected to be the leading end as IMD 114 is advanced through a delivery tool, such as a catheter, and placed against a targeted pacing site.

Electrodes 162 and 164 form an anode and cathode pair for bipolar cardiac pacing and sensing. In other examples, IMD 114 may include two or more ring electrodes, two tip electrodes, and/or other types of electrodes exposed along housing 150 for delivering electrical stimulation to a patient's heart and sensing at least one cardiac electrical signal. Tip electrode 164 is shown as a relatively flat button electrode. In other examples, tip electrode 164 may be a tissue piercing electrode having a helical or straight shaft, for example, configured to be advanced into cardiac tissue. Electrodes 164 may be positioned against or in operative proximity of the ventricular myocardium for sensing a ventricular electrical signal including T-wave signals used for determining a metric of changes in repolarization. In other examples, tip electrode 164 may be a tissue piercing electrode that may be advance into cardiac tissue in the vicinity of the ventricular conduction system to deliver conduction system pacing. Electrodes 162 and 164 may be, without limitation, titanium, platinum, iridium or alloys thereof and may include a low polarizing coating, such as titanium nitride, iridium oxide, ruthenium oxide, platinum black, among others. Electrodes 162 and 164 may be positioned at locations along IMD 114 other than the locations shown.

Housing 150 is formed from a biocompatible material, such as a stainless steel or titanium alloy. In some examples, the housing 150 may include an insulating coating. Examples of insulating coatings include parylene, urethane, PEEK, or polyimide, among others. The entirety of the housing 150 may be insulated, but only electrodes 162 and 164 uninsulated. Electrode 164 may serve as a cathode electrode and be coupled to internal circuitry, e.g., a pacing pulse generator and cardiac electrical signal sensing circuitry, enclosed by housing 150 via an electrical feedthrough crossing housing 150. Electrode 162 may be formed as a conductive portion of housing 150 defining a ring electrode that is electrically isolated from the other portions of the housing 150 as generally shown in FIG. 4. In other examples, the entire periphery of the housing 150 may function as an electrode that is electrically isolated from tip electrode 164, instead of providing a localized ring electrode such as anode electrode 162. Electrode 162 formed along an electrically conductive portion of housing 150 serves as a return anode during pacing and sensing.

The housing 150 includes a control electronics subassembly 152, which houses the electronics for sensing cardiac signals, producing pacing pulses and controlling therapy delivery and other functions of IMD 114 as described herein. Housing 150 further includes a battery subassembly 160, which provides power to the control electronics subassembly 152. Battery subassembly 160 may include one or more rechargeable or non-rechargeable batteries.

IMD 114 may include a set of fixation tines 166 to secure IMD 114 to patient tissue, e.g., by actively engaging with the ventricular endocardium and/or interacting with the ventricular trabeculae. Fixation tines 166 are configured to anchor IMD 114 to position electrode 164 in operative proximity to a targeted tissue for sensing cardiac electrical signals and delivering therapeutic electrical stimulation pulses. Numerous types of active and/or passive fixation members may be employed for anchoring or stabilizing pacemaker 14 in an implant position. IMD 114 may optionally include a delivery tool interface 158. Delivery tool interface 158 may be located at the proximal end 104 of IMD 114 and is configured to connect to a delivery device, such as a catheter, used to position IMD 114 at an implant location during an implantation procedure, for example within a heart chamber.

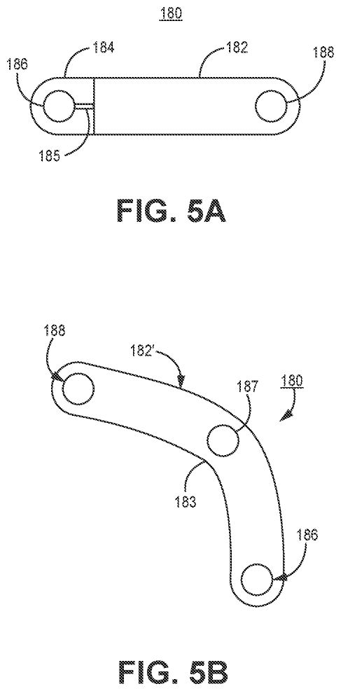

FIGS. 5A and 5B are conceptual diagrams of other examples of leadless medical devices that may be configured to sense at least one cardiac electrical signal and determine a metric of changes in repolarization according to the techniques disclosed herein. FIG. 5A is a conceptual diagram of sensing device 180. Sensing device 180 may be a cardiac monitoring device configured to sense at least one cardiac electrical signal that may be used for determining a metric of changes in repolarization of the myocardium for assessing a patient's risk of a cardiac event. Sensing device 180 includes a housing 182 that forms a hermetic seal that protects components within sensing device 180. Housing 182 may be formed of a conductive material, such as stainless steel or titanium alloy or other biocompatible conductive material or a combination of conductive and non-conductive materials. The housing 182 encloses one or more components, which may include one or more processors, memory, a transceiver, and sensing circuitry.

A header 184 is coupled to housing 182 for carrying electrode 186 and insulating electrical connections between electrode 186 and a sensing circuit enclosed in housing 182. Electrode 186 can be exposed on a surface of header 184. Header 184 encloses or encapsulates an electrical feedthrough 185 that extends from electrode 186 across housing 182 and electrically couples electrode 186 to the sensing circuitry enclosed by housing 182. A second electrode 188 may be formed as an uninsulated portion of housing 182 and serves as a ground or reference electrode. In some examples, the housing 182 may include an insulating coating. The entirety of the housing 182 may be insulated, but only electrode 188 uninsulated. Examples of insulating coatings include parylene, urethane, PEEK, or polyimide, among others. In other examples, an insulating coating of housing 182 is not provided, and all of housing 182 may function as an electrode 188. Electrodes 186 and 188 may be, without limitation, titanium, platinum, iridium or alloys thereof. In FIG. 5A, housing 182 is generally rectangular with electrodes 186 and 188 positioned near opposing ends of housing 182. Electrodes 186 and 188 may be positioned approximately 2 to 5 cm apart in some examples for acquiring a cardiac electrical signal that is received by sensing circuitry within housing 182. The cardiac electrical signal may be passed to processing circuitry enclosed by housing 182 for processing and analysis according to the techniques disclosed herein for determining a metric of change in repolarization measurements indicative of the patient's risk of a cardiac event. Sensing device may include a communication or telemetry circuit for transmitting a signal, e.g., by radio frequency signals, tissue conduction communication (TCC) or other communication protocols, in response to determining that the metric meets a risk threshold associated with the cardiac event.

FIG. 5B is a conceptual diagram of an alternative example of sensing device 180. In this example, housing 182′may be non-linear, angular housing including a curve or bend 183. Housing 182′may carry three electrodes 186, 187 and 188 to provide multiple sensing electrode vectors. Electrodes 186 and 188 may be carried at or near opposing ends of housing 182′, and a third electrode 187 may be located between electrodes 186 and 188. Electrode 187 may be located at housing bend 183 such that one sensing electrode vector between electrodes 188 and 187 is approximately horizontal (or extending in one direction) and another sensing electrode vector between electrodes 186 and 187 is approximately vertical (or extending in a second direction approximately orthogonal to the first direction). Electrodes 186, 187 and 188 may be equally spaced, e.g., at 2 to 8 centimeters apart (with no limitation intended). The electrode spacing between electrodes 186, 187 and 188 may vary between examples. For instance, without any limitation intended, electrodes 186 and 188 may be spaced apart approximately 1 inch to approximately 6 inches. In one example, the spacing between electrodes 186 and 188 is at least approximately 4 centimeters and up to approximately 10 centimeters with electrode 187 positioned between electrodes 186 and 188. In other examples, electrodes 186, 187 and 188 may be unequally spaced from each other such that one sensing electrode vector between electrode 187 and one of electrodes 188 or 186 has a greater inter-electrode distance than the other sensing electrode vector between electrode 187 and the other of electrodes 186 and 188.

Electrodes 186 and 188 may be electrically isolated from housing 182′and electrically coupled to a circuitry enclosed by housing 182′via an electrical feedthrough crossing the wall of housing 182′. Electrode 187 may be electrically coupled to housing 182′and serve as a ground or return electrode coupled to sensing circuitry enclosed by housing 182′. Housing 182′may be an electrically conductive housing having an insulating coating with electrode 187 being an uninsulated, exposed portion of conductive housing 182′. The angular housing 182′and electrodes 186, 187 and 188 is one example of a sensing device 180 that includes multiple sensing vectors. Other housing and electrode arrangements are conceivable that would provide multiple sensing vectors to enable processing circuitry to receive one cardiac electrical signal or two cardiac electrical signals that can be used for determining a metric of changes in repolarization of the myocardium as described herein.

Sensing device 180 of FIG. 5B may obtain cardiac electrical signals using a sensing electrode vector between electrodes 186 and 187 and between electrodes 188 and 187. In other examples, sensing device 180 may be configured to select one sensing electrode vector for sensing T-waves for analyzing changes in myocardial repolarization. Another electrode pair may be used for communication (e.g., transmitting or receiving a TCC signal to/from another medical device). Sensing device 180 configured to sense one cardiac electrical signal as shown in FIG. 5A or multiple cardiac electrical signals as shown in FIG. 5B may obtain T-wave signals that are analyzed by sensing device 180. When a metric of changes in repolarization is determined to meet a risk threshold based on the processing and analysis of T-wave signals from one cardiac electrical signal or up to two cardiac electrical signals, sensing device 180 may transmit a notification that can be received by another medical device, e.g., IMD 14 or external device 40 (shown in FIG. 1A). In some examples, sensing device 180 obtains T-wave signals that can be transmitted to another device, e.g., external device 40 or IMD 14 as shown in FIG. 1A, for performing the processing and analysis of the T-wave signals required to determine a metric of changes in repolarization for assessing a cardiac event risk of a patient. The processing and analysis of up to two cardiac electrical signals according to the techniques disclosed herein may be performed cooperatively between sensing device 180 or other cardiac monitoring device, such as the LINQ™ Insertable Cardiac Monitor (Medtronic, Inc., Dublin, Ireland) and the processing circuitry of another implanted or external device, such as the CARELINK SMARTSYNC™ Patient Monitor (Medtronic, Inc., Dublin, Ireland) or other remote or clinic-based patient monitoring system.

FIG. 6 is a conceptual diagram of a medical device configured to perform the techniques disclosed herein. FIG. 6 is described in conjunction with IMD 14 of FIGS. 1A-2C for the sake if illustration. It is to be understood however that the various components and circuitry described to perform the functionality disclosed herein may be implemented in other implantable or external devices (e.g., wearable or bedside devices) configured to determine a metric of changes in repolarization using up to two cardiac electrical signals. The electronic circuitry enclosed within the IMD housing 15 (shown schematically as an electrode in FIG. 6) may include software, firmware and/or hardware that cooperatively monitor cardiac electrical signals, determine when an electrical stimulation therapy is necessary, and deliver therapy as needed according to programmed therapy delivery algorithms and control parameters. IMD 14 may be coupled to a lead, such as lead 16 carrying electrodes 24, 26, 28, and 30, for sensing cardiac electrical signals and delivering electrical stimulation pulses to the patient's heart. As described above, in other examples electrodes used for receiving cardiac electrical signals may include or be exclusively housing-based electrodes, e.g., as shown in FIGS. 4, 5A and 5B.

IMD 14 includes a control circuit 80, memory 82, therapy delivery circuit 84, cardiac electrical signal sensing circuit 86, and telemetry circuit 88. A power source 98 provides power to the circuitry of IMD 14, including each of the components 80, 82, 84, 86, and 88 as needed. Power source 98 may include one or more energy storage devices, such as one or more rechargeable or non-rechargeable batteries. The connections between power source 98 and each of the other components 80, 82, 84, 86 and 88 are to be understood from the general block diagram of FIG. 6 but are not shown for the sake of clarity. For example, power source 98 may be coupled to one or more charging circuits included in therapy delivery circuit 84 for charging holding capacitors included in therapy delivery circuit 84 that are discharged at appropriate times under the control of control circuit 80 for producing electrical pulses according to a therapy protocol. Power source 98 is also coupled to components of cardiac electrical signal sensing circuit 86, such as sense amplifiers, analog-to-digital converters, switching circuitry, etc. as needed.

The circuits shown in FIG. 6 represent functionality included in IMD 14 and may include any discrete and/or integrated electronic circuit components that implement analog and/or digital circuits capable of producing the functions attributed to IMD 14 herein. Functionality associated with one or more circuits may be performed by separate hardware, firmware and/or software components, or integrated within common hardware, firmware and/or software components. For example, cardiac electrical signal sensing and analysis for detecting arrhythmia may be performed cooperatively by sensing circuit 86 and control circuit 80 and may include operations implemented in a processor or other signal processing circuitry included in control circuit 80 executing instructions stored in memory 82 and control signals such as blanking and timing intervals and sensing threshold amplitude signals sent from control circuit 80 to sensing circuit 86.

Control circuit 80 may include hardware configured to perform subroutines of signal processing and analysis techniques disclosed herein to reduce the processing burden associated with firmware and/or software execution of processing routines. For example hardware subroutines (HSRs) may be implemented in control circuit 80 to perform specific processing functions such as dedicated math operations, which may include any of sum, absolute value, difference, extrema, histogram counts, signal filtering (e.g., biquad filter, difference filter or other filters), etc. These HSRs could be called by control circuit firmware when processing and analyzing a cardiac signal for detecting arrhythmia and/or determining T-wave loops, repolarization measurements, changes in repolarization measurements, and a metric of changes in repolarization measurements. These HSRs can unload the processing burden associated with firmware and/or software processing to reduce current drain of power source 98 and thereby extend the useful life of IMD 14.

The various circuits of IMD 14 may include an application specific integrated circuit (ASIC), an electronic circuit, a processor (shared, dedicated, or group) and memory that execute one or more software or firmware programs, a combinational logic circuit, state machine, HSR, or other suitable components or combinations of components that provide the described functionality. The particular form of software, hardware and/or firmware employed to implement the functionality disclosed herein will be determined primarily by the particular system architecture employed in the medical device and by the particular sensing, detection and therapy delivery methodologies employed by the medical device. Providing software, hardware, and/or firmware to accomplish the described functionality in the context of any modern medical device system, given the disclosure herein, is within the abilities of one of skill in the art.

Memory 82 may include any volatile, non-volatile, magnetic, or electrical non-transitory computer readable storage media, such as random access memory (RAM), read-only memory (ROM), non-volatile RAM (NVRAM), electrically-erasable programmable ROM (EEPROM), flash memory, or any other memory device. Furthermore, memory 82 may include non-transitory computer readable media storing instructions that, when executed by one or more processing circuits, cause control circuit 80 and/or other medical device components to perform various functions attributed to IMD 14 or those IMD components. The non-transitory computer-readable media storing the instructions may include any of the media listed above.

Control circuit 80 communicates, e.g., via a data bus, with therapy delivery circuit 84 and sensing circuit 86 for sensing cardiac electrical signals, detecting cardiac rhythms, and controlling delivery of cardiac electrical stimulation therapies in response to sensed cardiac signals. To provide cardiac signal sensing and optional therapy delivery, therapy delivery circuit 84 and sensing circuit 86 are electrically coupled to electrodes 24, 26, 28, 30 carried by lead 16 and the housing 15, which may function as a common or ground electrode or as an active can electrode for delivering CV/DF shock pulses or cardiac pacing pulses.

Cardiac electrical signal sensing circuit 86 (also referred to herein as “sensing circuit” 86) may be selectively coupled to electrodes 28, 30 and/or housing 15 in order to monitor electrical activity of the patient's heart. Sensing circuit 86 may additionally be selectively coupled to defibrillation electrodes 24 and/or 26 for use in a sensing electrode vector together or in combination with one or more of electrodes 28, 30 and/or housing 15. Sensing circuit 86 may be enabled to receive cardiac electrical signals from at least one sensing electrode vector selected from the available electrodes 24, 26, 28, 30, and housing 15 in some examples. At least two, three or more cardiac electrical signals from two, three or more different sensing electrode vectors may be received simultaneously by sensing circuit 86 in some examples to be used for determining a heart rate, detecting arrhythmia, and performing T-wave analysis for cardiac event risk assessment. Sensing circuit 86 may monitor one or more cardiac electrical signals for sensing R-waves attendant to intrinsic ventricular myocardial depolarizations, T-waves attendant to ventricular myocardial repolarizations, and in some examples P-waves attendant to atrial myocardial depolarizations. In some examples, sensing circuit 86 may be configured to sense two cardiac electrical signals simultaneously to provide up to two cardiac electrical signals to control circuit 80 for T-wave analysis as described below in conjunction with the accompanying flow charts and diagrams.

As such, sensing circuit 86 may include one or more sensing channels that may each be selectively coupled to as sensing electrode vector via switching circuitry included in sensing circuit 86. Each sensing channel may include dedicated and/or shared sensing channel components configured to amplify, filter and digitize the cardiac electrical signal received from selected electrodes coupled to the respective sensing channel to improve the signal quality for sensing cardiac depolarization and repolarization signals, e.g., R-waves and T-waves. A sensing channel may include a pre-filter and amplifier circuit 83. Pre-filter and amplifier circuit 83 may include a high pass filter to remove DC offset, e.g., a 2.5 to 5 Hz high pass filter, or a wideband filter having a bandpass of 2.5 Hz to 100 Hz or narrower to remove DC offset and high frequency noise. Pre-filter and amplifier circuit 83 may further include an amplifier to amplify the raw cardiac electrical signal passed to analog-to-digital converter (ADC) 85. ADC 85 may pass a multi-bit, digital ECG signal (or electrogram (EGM) signal when the sensing electrodes are implanted inside the heart) to control circuit 80 for processing and analysis. The digital cardiac electrical signal received from ADC 85 may be buffered in memory 82 for subsequent processing and analysis. In some examples, segments of the digital cardiac electrical signal sensed during T-wave windows are buffered for processing and analysis as described below.

The digital signal from ADC 85 may be passed to rectifier and amplifier circuit 87, which may include a rectifier, bandpass filter, and amplifier for passing a cardiac signal to signal detector 89. Signal detector 89 may include a sense amplifier or other detection circuitry that compares the incoming rectified, cardiac electrical signal to a sensing threshold, which may be an auto-adjusting threshold. For example, when the incoming signal crosses an R-wave sensing threshold, the signal detector 89 may produce a ventricular sense signal (Vsense) that is passed to control circuit 80 to mark the timing of the sensed R-wave. Control circuit 80 may use the Vsense signal to apply a T-wave window to the incoming digitized cardiac electrical signal received from ADC 85 for obtaining T-waves for analysis as described below. In various examples, signal detector 89 may receive the digital output of ADC 85 for sensing R-waves, P-waves and/or T-waves by a comparator, morphological signal analysis of the digital signal or other signal detection techniques. The Vsense signals passed from signal detector 89 to control circuit 80 may also be used for scheduling ventricular pacing pulses delivered by therapy delivery circuit 84, determining a heart rate, and detecting arrhythmias. Control circuit 80 may provide sensing control signals to sensing circuit 86, e.g., sensing threshold adjustment parameters, sensitivity, and various blanking and refractory intervals applied to the cardiac electrical signal for controlling sensing of R-waves, P-waves and/or T-waves.

Control circuit 80 may include timing circuitry configured to control various timers and/or counters used in setting various intervals and windows used in sensing cardiac signals, determining time intervals between received Vsense signals, performing cardiac signal analysis and controlling the timing of electrical stimulation pulses (e.g. cardiac pacing pulses and/or CV/DF shocks) generated by therapy delivery circuit 84. The timing circuitry may start a timer in response to receiving Vsense signals from sensing circuit 86 for timing the RRIs between consecutively received Vsense signals, start a T-wave window, a pacing escape interval, and/or other timing control intervals.

Control circuit 80 may include arrhythmia detection circuitry configured to analyze RRIs received from the timing circuitry and cardiac electrical signals received from sensing circuit 86 for detecting arrhythmia. Control circuit 80 may be configured to detect asystole, long ventricular pauses, tachyarrhythmia and/or other cardiac arrhythmias based on sensed cardiac electrical signals meeting respective asystole, long pause, tachyarrhythmia detection or other criteria. For example, when a threshold number of ventricular sensed event signals from one sensing channel 83 or 85 each occur at a sensed event interval (RRI) that is less than a tachyarrhythmia detection interval, control circuit 80 may detect VT/VF. An RRI that is less than the tachyarrhythmia detection interval is referred to as a “tachyarrhythmia interval.” In some examples, a tachyarrhythmia detection based on the threshold number of tachyarrhythmia intervals (NID) being reached may be confirmed or rejected based on morphology analysis of a cardiac electrical signal.

As an example, the NID to detect VT may require that the VT interval counter reaches 18 VT intervals, 24 VT intervals, 32 VT intervals or other selected NID. In some examples, the VT intervals may be required to be consecutive intervals, e.g., 18 out of 18, 24 out of 24, or 32 out of 32 or 100 out of the most recent 100 consecutive RRIs. The NID required to detect VF may be programmed to a threshold number of X VF intervals out of Y consecutive RRIs. For instance, the NID required to detect VF may be 18 VF intervals out of the most recent 24 consecutive RRIs, 30 VF intervals out 40 consecutive RRIs, or as high as 120 VF intervals out of 160 consecutive RRIs as examples. When a VT or VF interval counter reaches a respective NID, a ventricular tachyarrhythmia may be detected by control circuit 80. The NID may be programmable and range from as low as 12 to as high as 120, with no limitation intended. VT or VF intervals may reach a respective NID when detected consecutively or non-consecutively out of a specified number of most recent RRIs. In some cases, a combined VT/VF interval counter may count both VT and VF intervals and detect a tachyarrhythmia episode based on the fastest intervals detected when a specified NID is reached.

Control circuit 80 may be configured to perform other signal analysis for determining if other detection criteria are satisfied before detecting VT or VF based on an NID being reached, such as R-wave morphology criteria, onset criteria, stability criteria and noise and oversensing rejection criteria. To support these additional analyses, sensing circuit 86 may pass a digitized cardiac electrical signal to control circuit 80 for detecting and discriminating heart rhythms.

In some examples, control circuit 80 may adjust tachyarrhythmia detection algorithms or control parameters in response to a metric of changes in repolarization measurements meeting a risk threshold. Control circuit 80 may turn on VT and/or VF detection, decrease an NID, adjust a tachyarrhythmia threshold interval, or otherwise enable tachyarrhythmia detection to be more sensitive and/or faster when the patient is deemed to be at risk of a cardiac event based on analysis of T-waves as described herein. In this way, ATP and/or CV/DF shocks can be promptly delivered when the patient is expected to be at higher risk of a cardiac event such as sudden cardiac death.

Therapy delivery circuit 84 includes at least one charging circuit 94, including one or more charge storage devices such as one or more high voltage capacitors for generating high voltage shock pulses for treating VT/VF. Charging circuit 94 may include one or more low voltage capacitors for generating relatively lower voltage pulses, e.g., for cardiac pacing therapies. Therapy delivery circuit 84 may include switching circuitry 95 that controls when the charge storage device(s) are discharged through an output circuit 96 across a selected pacing electrode vector or CV/DF shock vector.

In response to detecting VT/VF, control circuit 80 may schedule a therapy and control therapy delivery circuit 84 to generate and deliver the therapy, such as ATP and/or CV/DF shock(s). Therapy can be generated by initiating charging of high voltage capacitors of charging circuit 94. Charging is controlled by control circuit 80 which monitors the voltage on the high voltage capacitors, which is passed to control circuit 80 via a charging control line. When the voltage reaches a predetermined value set by control circuit 80, a logic signal is generated on a capacitor full line and passed to therapy delivery circuit 84, terminating charging. A CV/DF pulse is delivered to the heart under the control of control circuit 80 by an output circuit 96 of therapy delivery circuit 84 via a control bus. The output circuit 96 may include an output capacitor through which the charged high voltage capacitor is discharged via switching circuitry, e.g., an H-bridge, which determines the electrodes used for delivering the cardioversion or defibrillation pulse and the pulse wave shape. Therapy delivery circuit 84 may be configured to deliver electrical stimulation pulses for inducing tachyarrhythmia, e.g., T-wave shocks or trains of induction pulses, upon receiving a programming command from external device 40 (FIG. 1A) during ICD implant or follow-up testing procedures.

In some examples, the high voltage therapy circuit configured to deliver CV/DF shock pulses can be controlled by control circuit 80 to deliver pacing pulses, e.g., for delivering ATP, post shock pacing pulses, bradycardia pacing pulses or asystole pacing pulses. Therapy delivery circuit 84 may be configured to generate and deliver cardiac pacing pulses using the high voltage capacitor(s) that are chargeable to a shock voltage amplitude by charging the high voltage capacitor(s) to a relatively lower voltage corresponding to a cardiac pacing pulse amplitude for capturing and pacing the ventricular myocardium. Therapy delivery circuit 84 may include a low voltage therapy circuit including one or more separate or shared charging circuits, switch circuits and output circuits for generating and delivering relatively lower voltage pacing pulses for a variety of pacing needs. Charging of capacitors to a programmed pulse amplitude and discharging of the capacitors for a programmed pulse width may be performed by therapy delivery circuit 84 according to control signals received from control circuit 80 for delivering cardiac pacing pulses. As described above, timing circuitry included in control circuit 80 may include various timers or counters that control when cardiac pacing pulses are delivered. The microprocessor of control circuit 80 may set the amplitude, pulse width, polarity or other characteristics of cardiac pacing pulses, which may be based on programmed values stored in memory 82.

When control circuit 80 determines that a risk threshold is met by a metric of changes in repolarization measurements based on T-wave signal analysis as described below, control circuit 80 may control therapy delivery circuit to adjust a therapy. Ventricular pacing (e.g., high rate pacing), CRT, or other pacing therapy may be delivered or adjusted to reduce the likelihood of an onset of a tachyarrhythmia or other life-threatening cardiac event. In other examples, depending on the therapy delivery capabilities of the medical device system performing the techniques disclosed herein, vagus nerve stimulation, drug delivery or other therapies may be delivered. In some cases, telemetry circuit 88 may transmit a signal to another implanted or external device in response to detected changes in repolarization measurements to trigger a therapy delivery or instruct the patient to take a medication or seek medical attention.

Control parameters utilized by control circuit 80 for sensing cardiac event signals, detecting arrhythmias, and controlling therapy delivery may be programmed into memory 82 via telemetry circuit 88. Telemetry circuit 88 includes a transceiver and antenna for communicating with external device 40 (shown in FIG. 1A) using RF communication or other communication protocols as described above. Under the control of control circuit 80, telemetry circuit 88 may receive downlink telemetry from and send uplink telemetry to external device 40. Telemetry circuit 88 may transmit a notification in response to control circuit 80 determining that a metric of changes in repolarization meets a risk threshold in order to notify the patient or a clinician that medical attention or intervention may be warranted.

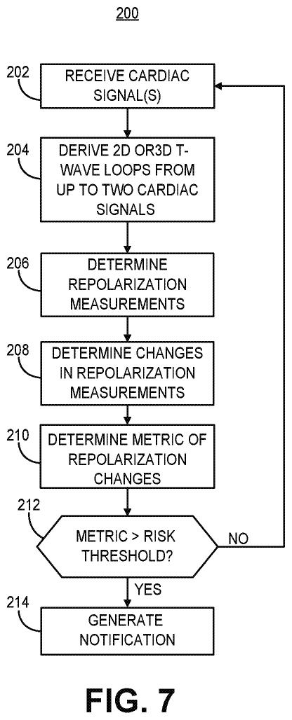

FIG. 7 is a flow chart 200 of a method performed by a medical device for determining a metric of cardiac repolarization changes (also referred to herein as a “metric of repolarization changes”) for predicting risk of a cardiac event, such as sudden cardiac death. For the sake of illustration, the process of flow chart 200 and other flow charts and diagrams presented herein are described as being performed by processing circuitry included in an IMD, e.g., by control circuit 80 of IMD 14. It is to be understood, however, that the techniques may be performed by processing circuitry of an external device, e.g., processor 52 of external device 40, or other computing device or processing circuitry of multiple devices, e.g., IMD 14 and external device 40, configured to operate cooperatively to perform the methods disclosed herein.

At block 202, control circuit 80 receives up to two cardiac electrical signals for T-wave signal analysis. The cardiac electrical signal(s) received at block 202 for T-wave signal analysis may include one or two ECG signals sensed from electrodes implanted outside the heart, e.g., subcutaneously, submuscularly, or substernally. Additionally or alternatively, the cardiac electrical signal(s) received at block 202 may include one or two EGM signals sensed from electrodes implanted in or on the patient's heart. In other examples, the processing circuitry receiving the cardiac electrical signal(s) for T-wave analysis at block 202 may receive ECG signals from surface electrodes placed on the patient's body.

It is recognized that processing circuitry configured to receive up to two cardiac electrical signals for performing T-wave analysis and determining a metric of repolarization changes may receive additional cardiac electrical signals for other medical device functions. For example, processing circuitry configured to perform the techniques disclosed herein may receive an atrial EGM signal and/or other ECG or EGM signals used for sensing R-waves, P-waves, detecting arrhythmias, determining heart rate, etc. However, the cardiac electrical signals received by the processing circuitry, e.g., control circuit 80, for performing T-wave analysis for determining a metric of repolarization changes consist of up to two cardiac electrical signals.

At least one cardiac electrical signal received at block 202 may be sensed from a sensing electrode vector in a substantially horizontal plane of the patient. A “substantially horizontal plane” may be a plane of the patient that is less than 45 degrees from a horizontal plane of the patient. For example, with reference to FIG. 1A, a sensing electrode vector between pace/sense electrode 28 or pace/sense electrode 30 and housing 15 may be used for sensing a first cardiac electrical signal. With reference to FIG. 3, a first cardiac electrical signal may be received at block 202 from a sensing electrode vector between any of the electrodes 124, 126, 128 or 130 carried by RV lead 117 and any of the electrodes 138 of CS lead 118 or housing 15. Depending on the implanted locations of electrodes available for sensing, when a single cardiac electrical signal is received, the sensing electrode vector may be a substantially sagittal sensing electrode vector that extends substantially in a horizontal plane of the patient between a relatively more posterior electrode and a relatively more anterior electrode.

In some examples, a second cardiac electrical signal is received from a second sensing electrode vector that may extend in a substantially frontal plane or a substantially horizontal plane. The second sensing electrode vector may extend approximately orthogonal to the first cardiac electrical signal, e.g., more than 45 degrees relative to the first sensing electrode vector. For instance, when the first sensing electrode vector extends between a relatively more posterior electrode and a relatively more anterior electrode, the second cardiac electrical signal can be received from a second sensing electrode vector that is a relatively transverse sensing electrode vector extending in a horizontal plane of the patient between a relatively leftward electrode and a relatively rightward electrode. In other instances, the second cardiac electrical signal can be received from a second sensing electrode vector extending substantially in a frontal plane of the patient between a relatively superior electrode and a relatively inferior electrode. For example, with reference to FIG. 1A, a sensing electrode vector between sensing electrode 28 and CV/DF electrode 24 or CV/DF electrode 26 may be used for sensing a second cardiac electrical signal. With reference to FIG. 3, a second sensing electrode vector may extend between tip electrode 128 and either of CV/DF coil electrodes 124 or 126. Example sensing electrode vectors described here are illustrative in nature and are not intended to be limiting. The sensing electrode vector(s) used for receiving one or two cardiac electrical signals at block 202 will depend on a number of factors including the number and location of electrodes available for sensing ECG or EGM signals.

At block 204, control circuit 80 derives a 2D or a 3D T-wave loop for each one of multiple cardiac cycles of the one or two cardiac electrical signals received at block 202. The T-wave loops may be derived from multiple consecutive or non-consecutive cardiac cycles. The T-wave loops may be derived from multiple consecutive cardiac cycles over 10 seconds, 30 seconds, 1 minute, 2 minutes, 5 minutes, 10 minutes, 30 minutes or one hour as examples. In some examples the T-wave loops are derived for each consecutive cardiac cycle for a specified number of cardiac cycles or during a specified time interval, e.g., at least one minute. In other examples, the T-wave loops may be derived for every nth cardiac cycle, e.g., every other cardiac cycle, every third cardiac cycle or every fourth cardiac cycle, as examples. The cardiac cycles may be non-paced cardiac cycles such that changes in repolarization can be assessed during an intrinsic heart rhythm. However, in some cases the rhythm may be a paced rhythm. For example, atrial pacing may be delivered in a patient having sinus node dysfunction. In other examples, ventricular pacing may be delivered in a patient having atrioventricular block or other conduction abnormalities. It is recognized, however, that variations in myocardial repolarization during pacing may be different than during an intrinsic rhythm.

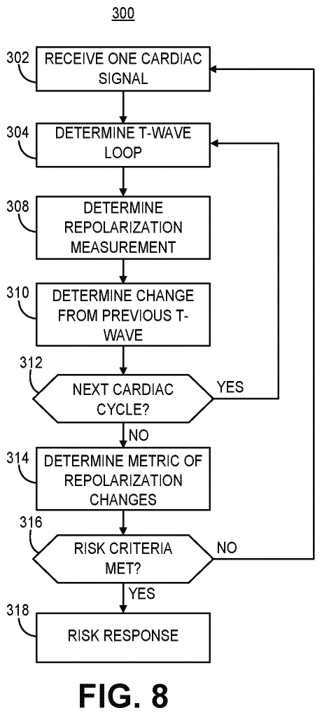

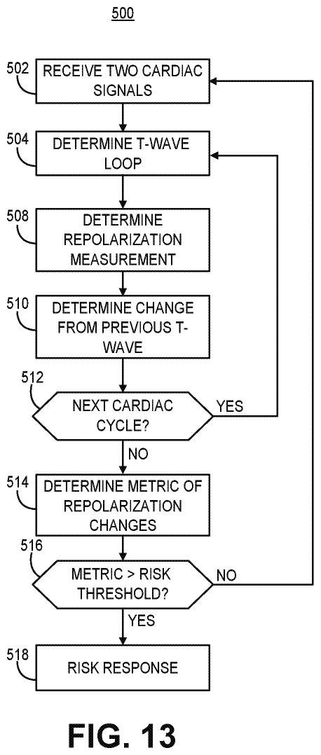

Each T-wave loop may be derived from a single cardiac electrical signal in some examples. Methods for deriving a 2D or 3D T-wave loop from a single cardiac electrical signal are described below, e.g., in conjunction with FIG. 8. In other examples, each T-wave loop may be derived from sensed cardiac electrical signals consisting of two cardiac electrical signals and may be 2D or 3D T-wave loops. Methods for deriving 2D or 3D T-wave loops from two cardiac electrical signals are described below, e.g., in conjunction with FIG. 13. T-wave loops may therefore be derived in 2D or 3D for use in determining a metric of repolarization changes from less than three cardiac electrical signals. In some cases, the T-wave loops are derived from fewer cardiac signals than the dimensionality of the T-wave loops. For example, the cardiac electrical signals analyzed by control circuit 80 may consist of two cardiac electrical signals for deriving 3D T-wave loops, or one cardiac electrical signal may be analyzed by control circuit 80 for deriving 2D or 3D T-wave loops. In some examples, two cardiac electrical signals may be used for deriving 2D T-wave loops. In each of these examples, less than three cardiac electrical signals are received by the processing circuitry for the purposes of determining a metric of repolarization changes.

At block 206, control circuit 80 may determine a repolarization measurement representative of each T-wave loop. As described below, control circuit 80 may determine a T-wave vector representative of the T-wave loop. The T-wave vector may be determined from the one or two cardiac electrical signals in a 2D or 3D polar coordinate system defined by a magnitude and angle(s). The repolarization measurement may be determined from the T-wave vector representative of the T-wave loop. For example, the repolarization measurement may be an angle relative to a coordinate system axis or plane that defines the location of the T-wave vector in the polar coordinate system. Examples of repolarization measurements determined from T-wave loops are described below.

At block 208, control circuit 80 determines changes between repolarization measurements, each corresponding to a respective cardiac cycle. For example, control circuit 80 may determine changes between a T-wave vector and a previous T-wave vector. The T-wave vectors may be derived in polar coordinates such that each T-wave vector is defined by at least one angle. In some examples, the changes between two repolarization measurements is the change between an angle of one T-wave vector and the angle of a previous T-wave vector relative to a polar coordinate axis (in 2D) or plane (in 3D). In some examples the angle of the T-wave vector may be determined as a weighted angle as described below. The determined change between two T-wave vectors can be determined as an angle between one T-wave vector derived in three dimensions from a first cardiac cycle of one or two cardiac electrical signals and a second T-wave vector derived in three dimensions from a second cardiac cycle (preceding or following the first cardiac cycle) of the one or two cardiac electrical signals.

The determined changes between successive repolarization measurements may be stored in memory 82. When a specified number of repolarization measurements have been determined or the T-wave vectors have been derived for multiple cardiac cycles of a specified time interval, control circuit 80 may determine a metric of repolarization changes from the repolarization measurements at block 210. The metric of repolarization changes may be determined as a quantitative metric of changes in amplitude, frequency or other component of the determined changes over time. The metric may be determined in the time domain or frequency domain. In some examples, the metric may be determined by performing a wavelet analysis of the time-based plot of repolarization changes. The metric of repolarization changes may be representative of a periodic change that occurs in the T-wave loops. The periodic change may be associated with sympathetic nervous activity and be representative of risk of a cardiac event.

At block 212, control circuit 80 may compare the metric of repolarization changes to a risk threshold. When the metric meets the risk threshold, e.g., is greater than the risk threshold, control circuit 80 may generate a notification at block 214 indicating a predicted risk of a cardiac event, such as sudden cardiac death. Telemetry circuit 88 may transmit the risk notification to a receiving device, which may be personal device, medical device programmer, remote patient monitoring system, another implanted medical device capable of delivering a therapy, or the like.

FIG. 8 is a flow chart 300 of a method for deriving repolarization measurements from a single cardiac electrical signal received by processing circuitry of a medical or computing device. For the sake of illustration, the process of flow chart 300 is described with reference to IMD 14 including control circuit 80. At block 302, control circuit 80 receives one cardiac electrical signal, e.g., an ECG or EGM signal, for processing and analysis for determining a metric of repolarization changes. The cardiac electrical signal may be received from a sensing electrode vector extending along a horizontal plane of the patient in some examples and may be a sagittal or transverse sensing electrode vector in some examples. However, the positions of the sensing electrodes for sensing the one cardiac electrical signal are not limited to a particular location or orientation.

At block 304, control circuit 80 may derive a 2D T-wave loop from the received cardiac electrical signal for a respective cardiac cycle of the one cardiac electrical signal. The T-wave loop may be derived in two dimensions from the received cardiac electrical signal by determining ordered pairs from sample points of the received cardiac electrical signal using a lag time between x and y values of the ordered pairs.

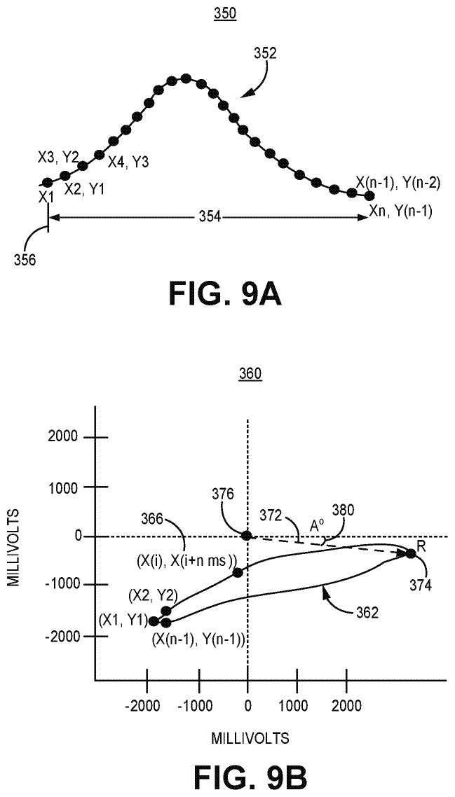

FIG. 9A is a diagram 350 of a T-wave 352 that may be sensed from a cardiac electrical signal during a T-wave window 354. The T-wave window 354 may be 150 to 400 ms in duration or about 200 to 300 ms in duration as examples. The T-wave window 354 may have a beginning time 356 at 200 to 400 ms or about 250 to 300 ms after a ventricular depolarization event, e.g., after a Vsense signal, the onset of a QRS waveform, an R-wave peak, a ventricular pacing pulse or other fiducial point of the QRS waveform. In some examples, the T-wave window 354 may have a beginning time 356 that is applied at a selected time interval following an atrial pacing pulse that may be known to conduct to the ventricles. In other examples, the T-wave 352 may be detected by control circuit 80, e.g., based on a threshold crossing, maximum peak amplitude of T-wave 352 or other waveform morphology analysis. T-wave window 354 may be applied to the received cardiac electrical signal having a beginning time 356 relative to the detected T-wave, e.g., relative to a threshold crossing or maximum peak amplitude or other fiducial point of the T-wave 352. T-wave 352 may be sampled to obtain sample points X1 through Xn at a desired sampling rate over T-wave window 354.

FIG. 9B is a diagram 360 of a T-wave loop 362 that may be generated by control circuit 80 from a single cardiac electrical signal. With continued reference to FIGS. 7, 8A and 8B, control circuit 80 may receive T-wave signal 352 in a cardiac electrical signal sensed from a sensing electrode vector during one cardiac cycle at block 302. Control circuit 80 may derive the 2D T-wave loop 362 from the T-wave signal 352 at block 304 of FIG. 8. The T-wave loop 362 may be derived from T-wave signal 352 using attractor theory in some examples.

In the example shown, control circuit 80 may generate T-wave loop 362 by obtaining ordered (x, y) pairs from the received T-wave signal 352. The x-coordinate of each ordered pair can be the amplitude of the ith point of T-wave 352, and the y-coordinate can be the amplitude of the i+1 point of T-wave 352, where each ith point i+1 point may be separated by a selected sample time difference, e.g., 0.5 ms, 1 ms, 2 ms, 4 ms, 5 ms, 8 ms, 10 ms, 16 ms, 20 ms, 32 ms or other selected time sample time difference. As shown in FIG. 9A, consecutive sample point amplitudes of T-wave 352 define each of the X(1) through X(n−1) values of the x-coordinates of the T-wave loop 362 shown in FIG. 9B. Each Y(1) through Y(n−1) amplitude, which correspond to the X(n+1) to X(n) sample points of T-wave 352, define the y-coordinates of the T-wave loop 362 in each respective (x, y) ordered pairs. Accordingly, each point on T-wave loop 362 may be defined by the (X(i), X(i+n ms)) ordered pair 366 where X(i) is the amplitude of the ith sample point of T-wave 312 (defining the x-coordinate of a point on T-wave loop 362), and X(i+n ms) is the amplitude of the sample point n ms after the ith sample point (defining the y-coordinate of a point on T-wave loop 362).

It is to be understood that the cardiac electrical signal may be sampled at a sensing sampling rate that is the same or different than the sampling rate corresponding to the time lag between T-wave sample points used to obtain the (X, Y) coordinate pairs from T-wave 352 for generating the T-wave loop 362. For example, the cardiac electrical signal may be sensed using a sampling rate of 128 to 1024 Hz. The n ms time lag between points selected for generating the T-wave loop 362 of FIG. 9B may be greater than, equal to or less than the sample time between sample points of the sensed cardiac electrical signal. For example, if the received cardiac electrical signal is sampled at 256 Hz with approximately 4 ms between sample points, the T-wave loop 362 may be generated using every sample point (a time lag of 4 ms between X and Y coordinate amplitudes), every other sample point (a time lag of 8 ms between X and Y coordinate amplitudes), every third sample point (a time lag of 12 ms between X and Y coordinate amplitude) etc. In some examples, X and Y coordinate amplitudes may be or interpolated between sample points of the sensed cardiac electrical signal. For example, the X and Y coordinate amplitudes of a given point on T-wave loop 362 may be separated by 2 ms on T-wave 352 when the cardiac electrical signal is sampled at 256 Hz, with amplitudes of x-and y-coordinates being interpolated at 2 ms intervals between sample points of the T-wave 352, e.g., by averaging or other interpolation methods.

Referring again to FIG. 7, control circuit 80 may determine a repolarization measurement from the T-wave loop at block 308. The repolarization measurement may be a quantitative representation of the T-wave loop, e.g., T-wave loop 362 shown in FIG. 9B. T-wave vector 372 (FIG. 9B) may be determined at block 308 of FIG. 8 as a representative measure of the cardiac repolarization for the current cardiac cycle. Control circuit 206 may determine the T-wave vector 372 from the 2D T-wave loop 362 as the vector extending from the origin 376 of the cartesian coordinate system to the point 374 on the T-wave loop 362 that is the greatest distance R from the origin 376. Control circuit 80 may determine T-wave vector 372 in polar coordinates defined by angle A 380 from the x-axis having magnitude R. The angle A 380 may be measured relative to the y-axis instead of the x-axis in other examples. Zero degrees can be defined as being aligned with the positive x-axis with increasing angles in the clockwise direction as in the example shown. In other examples, zero degrees may be defined as aligned with the negative x-axis, positive y-axis or negative y-axis with increasing angles in the clockwise or counterclockwise direction. When the cardiac electrical signal is sensed using a sensing electrode vector extending substantially horizontally in a sagittal plane, the y-axis may correspond to the sagittal plane, and the x-axis may correspond to the horizontal plane. The repolarization measurement of the cardiac cycle including T-wave 352 may be determined by control circuit 80 and buffered in memory 82 as the angle A 380 calculated relative to the x-or y-axis according to any of the examples given above, the magnitude R 374, and/or the product of the angle and the magnitude, A*R, in various examples.

In other examples, control circuit 80 may determine the repolarization measurement from T-wave loop 362 by computing a weighted angle measurement using the (X(i), X(i+n ms)) points of T-wave loop 362. Control circuit 80 may convert each point of T-wave loop 362 to polar coordinates defined by an angle “a” in the polar coordinate system e.g., the angle from the x-axis, and having a magnitude “r”. Control circuit 80 may compute the weighted angle measurement (WAM) by summing the product of each angle and magnitude of the T-wave loop points (WAM=Σa(i)*r(i) where i=1 to n−1 when a total of n−1 points define the T-wave loop 362 based on n sample points of T-wave 352). The WAM may be normalized by the summation of the magnitudes “r” of each T-wave loop point in some examples (WAM={Σa(i)*r(i)}/{Σr(i)} where i=1 to n−1). The WAM may be buffered in memory 82 as the repolarization measurement for the given cardiac cycle.

Other examples of a repolarization measurement that control circuit 80 may compute from a T-wave loop may include the area of the T-wave loop, the area of the T-wave loop projected in a 2D plane (when a 3D T-wave loop is determined), the total length of the perimeter of the T-wave loop, or the centroid of the T-wave loop. One or more repolarization measurements may be determined. In some examples, a combination of the repolarization measurements may be determined. A combination of multiple repolarization measurements may be determined, which may be a sum, weighted sum, product, difference and/or ratio or any other combination. In some examples, one (or a combination of) repolarization measurement(s) may be normalized by another (or combination of) repolarization measurement(s) to obtain a repolarization measurement representative of a T-wave loop.

At block 310, control circuit 80 may determine a change in the repolarization measurement from a previous repolarization measurement. The change may be a difference in the repolarization measurement, e.g., the difference in the WAM, the difference in A, difference in R, or difference in A*R, from a previous repolarization measurement, which may be the most recent preceding repolarization measurement. At block 312, control circuit 80 may determine if another cardiac cycle is available for determining a repolarization measurement of a next T-wave. A repolarization measurement and corresponding change from a previous repolarization measurement may be determined for a specified number of T-waves or for all T-waves (or every other T-wave, every third T-wave, etc.) that occur during a specified time period for assessing repolarization changes. When multiple repolarization measurements are determined from each T-wave loop, the change in the repolarization measurements may be determined for each repolarization measurement. In some examples, the changes in each of multiple repolarization measurements may be combined mathematically as a sum, ratio, product or other combination to obtain a change in repolarization measurements between two cardiac cycles.

When another cardiac cycle is available, control circuit 80 may return to block 304 to determine the 2D T-wave loop point coordinates from the received cardiac signal for the next T-wave. When a specified number of T-waves or specified time period of the cardiac signal have been evaluated, control circuit 80 may advance to block 314 to determine a metric of the determined repolarization measurement changes.

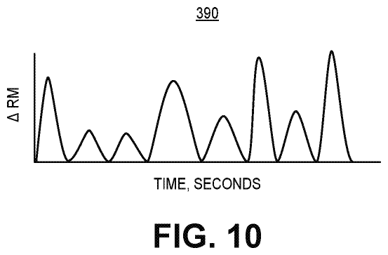

FIG. 10 is an illustrative plot 400 of determined changes in repolarization measurements (A RM) that may be accumulated in memory 82 over a specified time period or number of cardiac cycles. The repolarization measurement can have a periodic behavior due to periodicity of sympathetic activity. The change in the repolarization measurements over successive cardiac cycles can be increased in patients at risk of a clinically significant or life-threatening cardiac event. Control circuit 80 may be configured to determine a quantitative metric of the periodic changes in repolarization measurements at block 314 of FIG. 8. The metric may be determined in the time domain or the frequency domain. The metric may be a representative amplitude, such as the average peak amplitude, summation of sample points greater than a threshold value, or other value determined from the amplitudes of repolarization measurement changes.

In other examples, the metric of repolarization changes may be determined as a mean frequency, center frequency, or dominant frequency of the repolarization metric changes. In some examples, a wavelet transform of a plot of the repolarization metric changes over time may be performed by control circuit 206 and the maximum wavelet coefficient may be determined as the metric of repolarization changes. In other examples, phase rectified signal averaging may be applied to the repolarization measurement changes over time to obtain a maximum frequency or center frequency after phase rectified signal averaging. In some examples, a wavelet transformation of the repolarization measurement changes over time may be performed and the average wavelet coefficient for frequencies in a low frequency range, e.g., less than 0.5 Hz, less than 0.3 Hz, less than 0.2 Hz, or less than 0.1 Hz may be determined as the metric at block 314.

In another example, the metric of repolarization changes may be determined by determining variability in the average beat to beat differences of repolarization metrics determined over a specified number of consecutive cardiac cycles. For example, the difference between two consecutive repolarization measurements may be determined for each of 3, 5, 6, 8, 10, 20 or other specified number of cardiac cycles and averaged to determine an average beat to beat difference. This process may be repeated for the next specified number of cardiac cycles to determine the next average beat to beat difference. The variability of the successive average beat to beat differences may be determined as a metric of repolarization changes. The variability in successive average beat to beat differences may reflect a high variability or chaos in the repolarizations indicative of risk of a cardiac event.

In yet another example, the metric of repolarization change could be determined as a maximum slope of the repolarization changes plotted over time, a minimum slope of the repolarization changes over time, or the difference between the maximum and minimum slopes of the repolarization changes plotted over time. Patients with less compensatory mechanisms (vagal compensation) may have steeper transitions between repolarization measurements than patients at less risk for a cardiac event.

In the examples of FIGS. 9A, 9B and 10, the T-wave loop is determined in two dimensions from the cardiac electrical signal using (X(i), X(i+n ms)) ordered pairs determined from the single cardiac electrical signal. The metric of repolarization changes is determined from the repolarization measurement changes in the 2D T-wave loops. In other examples, a 3D T-wave loop may be derived from the single cardiac electrical signal for each of multiple cardiac cycles. To derive the 3D T-wave loop, cartesian coordinates in three dimensions may be determined as (X(i), X(i+n ms), X(i+m ms)) to define each T-wave loop point that can be plotted along the x-, y-and z-axes of a cartesian coordinate system. The third dimension of the cartesian coordinate, X(i+m ms) may be determined at m ms from the X(i) point of the T-wave signal (see FIG. 9A), where m may be equal to 2n (double the time lag of the X(i) point relative to the X(i+m ms) point). However, m may be any value different than or equal to n for extracting 3D cartesian coordinates from a single cardiac electrical signal for generating a 3D T-wave loop for a respective cardiac cycle of the single cardiac electrical signal.

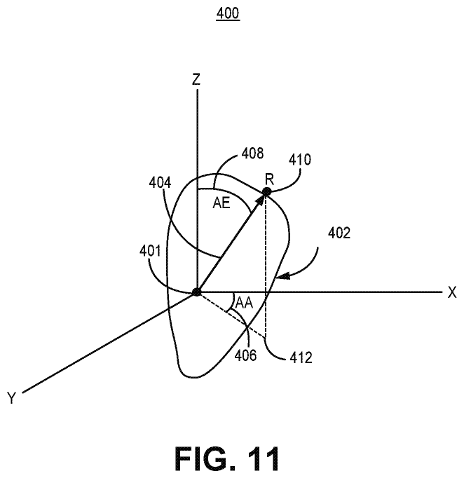

FIG. 11 is diagram 400 of an example 3D T-wave loop 402 that may be generated from a single cardiac electrical signal. Each point of the 3D T-wave loop 402 may be defined by cartesian coordinates determined from the signal cardiac electrical signal as (X(i), X(i+n ms), X(i+m ms)) as described above. A repolarization measurement may be determined from the T-wave loop 402. The points of the T-wave loop 402 may be converted to a polar coordinate system and a repolarization measurement may be determined from the T-wave loop 402. In some examples, one or more points of the T-wave loop can be converted to a polar coordinate system for determining the repolarization measurement.

For example, a representative T-wave vector 410 may be determined. T-wave vector 410 may be determined as the vector extending from the origin 401 to a point on the T-wave loop 402 that is the greatest distance from the origin 402. A repolarization measurement may be determined from the T-wave vector 410 as the angle of azimuth (AA) relative to the x-axis (or y-axis) of a projection 412 of T-wave vector 410 in the x-y plane. A repolarization measurement may be determined from the T-wave vector 410 as an angle of elevation (AE) 408 between the z-axis and the T-wave vector 410 (or between the x-y plane and the T-wave vector 410). A repolarization measurement may be determined as the magnitude R of the T-wave vector 410, a weighted AA (AA *R), a weighted AE (AE*R), the area of the T-wave loop 410, a distance from the point on the T-wave loop 402 that is nearest the origin 401 to the point on the T-wave loop 402 that is furthest from the origin 401, or a greatest distance between any two points of the T-wave loop 402 may be determined as various examples of a repolarization measurement that is representative of T-wave loop 402.

In some examples, T-wave vector 404 is determined as a unit vector defined by a weighted AA (WAA) and a weighted AE (WAE) from the points of the T-wave loop 402 converted to polar coordinates. The WAA may be computed as the summation WAA={Σaa(i)*r(i)}/{Σr(i)} where i=1 to n−2 for n T-wave sample points, aa(i) is the angle of azimuth of the ith point on T-wave loop 402, and r(i) is the magnitude of the ith point on T-wave loop 402 in polar coordinates. The WAE may be computed as the summation WAA={Σaa(i)*r(i)}/{Σr(i)} where i=1 to n−2), ae(i) is the angle of elevation of the ith point on T-wave loop 402, and r(i) is the magnitude of the ith point on T-wave loop 402 in polar coordinates. When the T-wave vector 404 is determined as a unit vector defined by WAA and WAE, the change in the repolarization measurement from one T-wave to another T-wave may be determined as the change in WAA, change in WAE, or the change in the angle between one T-wave vector and the next T-wave vector in three dimensions.

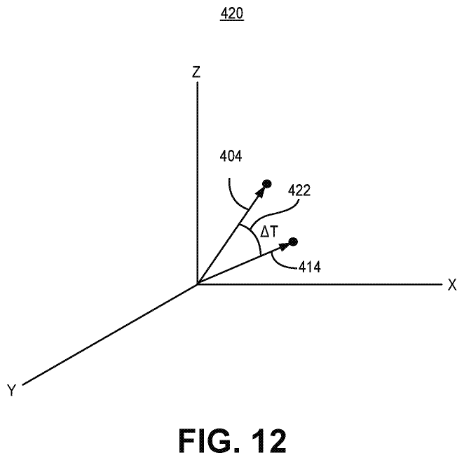

FIG. 12 is a diagram 420 of two T-wave vectors, each representative of a T-wave loop determined from a single cardiac cycle, that may be determined by processing circuitry of a medical device according to some examples. Diagram 420 includes the T-wave vector 404 shown in FIG. 11, which may be determined as a unit vector defined by a WAA and by a WAE. A second T-wave vector 414 may be determined by control circuit 80 for a subsequent cardiac cycle. The angle AT 422 between the two T-wave vectors 404 and 414 may be determined as the repolarization measurement change between two cardiac cycles. The angle AT 422 may be determined by computing the dot product of the T-wave vectors 404 and 414. The angle AT, or any other change determined between 3D T-wave vectors 404 and 414, may be stored over time for multiple cardiac cycles to obtain a time-based AT signal, e.g., analogous to the time-based ARM signal shown in FIG. 9. A quantitative metric of the time based AT (or more generally ARM signal for any aspect of change between successive 3D T-wave vectors) can be determined according to any of the examples given herein.

Returning to FIG. 8, at block 316, control circuit 80 may compare the metric of repolarization changes determined from either 2D or 3D T-wave loops to risk criteria, e.g., a risk threshold. The risk threshold may be established from empirical data from a population of patients. The risk threshold may be established from a population of patients that are known to have no history of cardiac events. The risk threshold may be established from a population of patients that are known survivors of a cardiac event, such as a myocardial infract. The risk threshold may be established from a population of patients that are known non-survivors of the cardiac event. The risk threshold may be established to be between an average metric from a population of patients that are known survivors of a cardiac and the average metric from a population of patients that are known non-survivors of a cardiac event. In other examples, the risk threshold may be established from empirical data from a population of patients with no history of a cardiac event and/or patients with a known history of the cardiac event.

In other examples, the risk threshold may be tailored to a patient. A baseline metric may be determined from the patient using a baseline cardiac electrical signal recorded from the patient. The metric of repolarization changes determined at a later time point may be compared to the baseline metric or a risk threshold established based on the baseline metric. For example, the metric of repolarization changes may be determined to be greater than the risk threshold when an increase in the metric from a baseline metric is greater than 10%, 20%, 30% or other threshold percentage increase, for example.

In still other examples, the metric of repolarization changes may meet risk criteria when it is the nth metric of n continuously increasing metrics of repolarization changes. For example, if the most recent three, five, eight or other threshold number of metrics of repolarization changes each represent an increase over a previous metric of repolarization changes, control circuit 80 may determine that the risk threshold is met. In another example, control circuit 80 may determine that the risk criteria is met when at least x metrics of depolarization represent an increase over a previous metric of repolarization changes within a given time period, e.g., within one hour, 24 hours, 48 hours, 72 hours or other time period. In another example, control circuit 80 may sum successive differences between metrics of repolarization changes and compare the sum of successive differences to a risk threshold. When the sum of successive differences meets the risk threshold, a continuously increasing metric of repolarization changes may indicate that the patient is at risk of a serious cardiac event.

When the metric of repolarization changes does not meet the risk criteria (“no” branch of block 316), control circuit 80 may return to block 302 to receive the cardiac electrical signal the next time that monitoring for the risk of a cardiac event is to be performed. The process of FIG. 8 may be performed continuously, once a day, once a week or other scheduled frequency. The process of flow chart 300 may be triggered in response to detecting an arrhythmia, an increase in a tachyarrhythmia burden, an increase in non-sustained tachyarrhythmia occurrences, or other changes in the cardiac rhythm that may be determined by control circuit 80 from one or more cardiac electrical signals received from sensing circuit 82.

When the metric of repolarization changes meets the risk criteria (“yes” branch of block 316), control circuit 80 may perform a response to determining that the risk threshold is met block 318. The response may include generating a risk notification that may be transmitted by telemetry circuit 88. The response may include delivering or adjusting a therapy. For example, control circuit 80 may control therapy delivery circuit 84 to deliver cardiac pacing at a pacing rate greater than the intrinsic ventricular rate and/or according to a pacing mode to promote a stable heart rhythm. The response may include adjusting a tachyarrhythmia detection method or control parameter. For example, control circuit 80 may turn on VT and/or VF detection, adjust one or more parameters used in detecting tachycardia or fibrillation to decrease the time required to detect a tachyarrhythmia, and/or adjust one or more detection control parameters to increase the sensitivity for detecting tachyarrhythmias so that ATP and/or CV/DF shocks may be delivered in a time-efficient manner when a tachyarrhythmia is detected.

FIG. 13 is a flow chart 500 of a method for determining a metric of repolarization changes for predicting risk of a cardiac event according to another example. The techniques described in conjunction with FIGS. 7-11 do not require receiving more than one cardiac electrical signal for determining the metric of repolarization changes. In other examples, the processing circuitry computing the metric of repolarization changes may receive up to two cardiac electrical signals. For the sake of illustration, the process of FIG. 13 is described as being performed by control circuit 80 of IMD 14. Though it is to be understood that the techniques may be performed by a different implantable device, an external computing device or cooperatively by processing circuitry of more than one implanted and/or external device.

At block 502, control circuit 80 receives two cardiac electrical signals. The cardiac electrical signals may be received from two sensing electrode vectors, which may be approximately orthogonal to each other in some examples. The two sensing electrode vectors may correspond to a horizontal plane of the patient and may consist of a sagittal sensing electrode vector and a transverse sensing electrode vector. The two sensing electrode vectors may include one sensing electrode vector in a substantially horizontal plane (sagittal or transverse) and one sensing electrode vector in a substantially vertical plane, e.g., in a frontal plane or a sagittal plane. It is recognized that depending on the number and locations of the implanted and/or external electrodes used in the two sensing electrode vectors, the sensing electrode vectors may extend in non-orthogonal relationships and may extend relatively diagonally as opposed to being substantially in a vertical or horizontal plane of the patient.

At block 504, control circuit 80 determines a T-wave loop from a cardiac cycle of the two cardiac signals. In some examples, a 2D T-wave loop is derived from the two cardiac signals by obtaining pairs of time-aligned sample points from the two cardiac electrical signals over a T-wave window. Each pair of time-aligned sample points from the two cardiac electrical signals can define an ordered pair in a cartesian coordinate system.

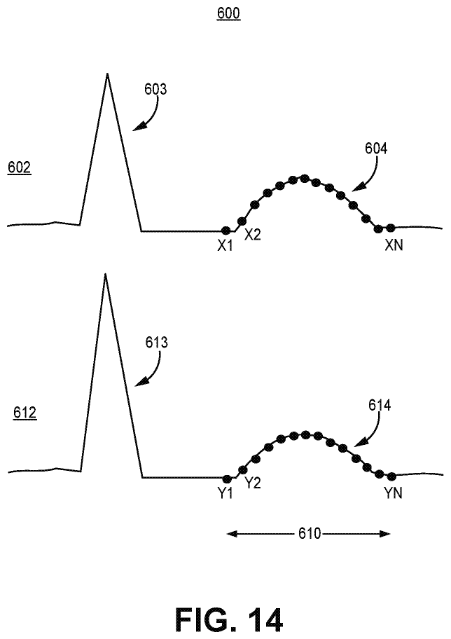

FIG. 14 is a diagram 600 of two cardiac electrical signals 602 and 612 that may be received by control circuit 80 for use in determining T-wave loops and a metric of repolarization changes from the T-wave loops. Points from each T-wave 604 and 614 may be sampled over a T-wave window 610 for obtaining X and Y pairs of sample point amplitudes from the respective T-waves 604 and 614. Each (X1, Y1), (X2, Y2) through (XN, YN), time-aligned sample point pair defines the x and y coordinates of a point on a 2D T-wave loop. As described above, the T-wave window 610 may have a beginning time set relative to a preceding R-wave 603 or 613 (e.g., the time of an R-wave sensing threshold crossing, R-wave maximum peak, etc.). In other examples, a T-wave 604 or 614 may be identified based on a threshold crossing, peak amplitude, or other identifiable feature of the T-wave 604 or 614 to enable control circuit 80 to set the T-wave window 610 that is applied to both cardiac electrical signals 602 and 612 for acquiring ordered (X, Y) pairs from the amplitudes of sample points of T-waves 604 and 614.

In other examples, control circuit 80 may derive a 3D T-wave loop from the two received cardiac electrical signals. The position of a T-wave loop point along a third axis of a 3D coordinate system may be determined from one or both of the received cardiac electrical signals 602 and 612. For example, a T-wave loop point defined by (X, Y, Z) coordinates may be determined having an x-coordinate from T-wave 604 of the first received cardiac electrical signal 602, a y-coordinate from T-wave 614 of the second received cardiac electrical signal 612 and a z-coordinate determined from a combination of the two cardiac electrical signals 602 and 612. For instance, each z-coordinate may be the sum, difference, product, quotient or other combination of the time-aligned sample points of the first cardiac electrical signal 602 and the second cardiac electrical signal 612. The z-coordinates may be determined from time-aligned sample points of the T-waves 604 and 614 or from a sample point of T-wave 604 that is shifted in time from the sample point of T-wave 614, e.g., by a lead time interval or a lag time interval. The lead time or lag time interval may be between 0.5 and 20 ms in various examples. For example, each T-wave loop point (X, Y, Z) may be defined in a three dimensional cartesian coordinate system as (X(i), Y(i), Z(i)) where Z(i) may be determined as w1*X(i+n ms)+w2*Y(i+m ms) where w1 and w2 may be weighting values that can be equal to 1 or any other fractional or integer value, n may be between −20 ms and +20 ms and may be equal to zero, and m may be between −20 ms and +20 ms and may be equal to zero.

Accordingly, in some examples, a third coordinate of each T-wave loop point may be determined from either one of the first cardiac electrical signal 602 or the second cardiac electrical signal 612 (when the weighting factor w1 or w2 is zero). For instance, the third coordinate may be determined as the sample point amplitude from either T-wave 604 or T-wave 614 that is leading or lagging the time-aligned x and y coordinate sample points by a lead time or a lag time interval. For example, each T-wave loop point (X, Y, Z) may be defined in a three dimensional cartesian coordinate system as (X(i), Y(i), X(i+n ms)), as an example. A variety of methods for deriving a third coordinate value from the two T-wave signals 604 and 614 may be conceived given the illustrative examples presented herein.

Returning to FIG. 13, after determining the T-wave loop in in two or three dimensions from received cardiac electrical signals consisting of two cardiac electrical signals, control circuit 80 may determine a repolarization measurement from the T-wave loop at block 508 according to any of the examples given herein. At block 510, control circuit 80 may determine a change in the repolarization measurement from a previous determined repolarization measurement. The process of determining the T-wave loop, determining a repolarization measurement from the T-wave loop, and determining a change in the repolarization measurement from a preceding repolarization measurement may be repeated for each cardiac cycle of multiple cardiac cycles. When all cardiac cycles have been analyzed as needed for determining the metric of repolarization changes, as determined at block 512, the metric of repolarization changes can be determined by control circuit 80 at block 514 according to any of the examples described herein.

At block 516, the metric of repolarization changes can be compared to a risk threshold by control circuit 80 to provide a risk response at block 518 when the metric of repolarization changes meets the risk threshold. As described above, an alert may be transmitted by telemetry circuit 88 and/or a cardiac electrical stimulation therapy may be delivered or adjusted in response to the metric of repolarization changes meeting the risk threshold. Additionally or alternatively, tachyarrhythmia detection function may be turned on or adjusted to provide earlier and/or more sensitive tachyarrhythmia detection.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

The following examples are a non-limiting list of clauses in accordance with one or more techniques of this disclosure.

### Example 1

A medical device, comprising: processing circuitry configured to: receive up to two cardiac electrical signals; for each of a plurality of cardiac cycles of the received cardiac electrical signal(s) consisting of the up to the two cardiac electrical signals: derive a T-wave loop in at least two dimensions; determine a repolarization measurement representative of the T-wave loop; and determine a change in the repolarization measurement from a previously determined repolarization measurement; determine a metric of the determined changes in the repolarization measurements; and determine that the metric meets a risk threshold associated with a cardiac event; and a telemetry circuit configured to transmit a risk notification in response to the metric meeting the risk threshold.

### Example 2

The medical device of Example 1, wherein the processing circuitry is further configured to derive the T-wave loop in at least two dimensions from a first cardiac electrical signal of the up to two cardiac electrical signals by determining a first coordinate in a first dimension and a second coordinate in a second dimension of each point of a plurality of points of the T-wave loop by: determining the first coordinate as a first amplitude of a first sample point of the first cardiac electrical signal; and determining the second coordinate as a second amplitude of a second sample point of the first cardiac electrical signal, the second sample point offset by a first time interval from the first sample point.

### Example 3

The medical device of Example 2, wherein the processing circuitry is further configured to derive the T-wave loop in three dimensions from the first cardiac electrical signal by determining a third coordinate in a third dimension of each point of the plurality of points of the T-wave loop as a third amplitude of a third sample point of the first cardiac electrical signal, the third sample point offset by a second time interval from the first sample point.

### Example 4

The medical device of Example 3, wherein the processing circuitry is further configured to determine the third amplitude of the third sample point offset by the second time interval from the first sample point where the second time interval is different than the first time interval.

### Example 5

The medical device of any of Examples 2-4, wherein the processing circuitry is further configured to derive the T-wave loop in three dimensions from the first cardiac electrical signal and a second cardiac electrical signal of the up to two cardiac electrical signals by determining a third coordinate in a third dimension of each point of the plurality of points of the T-wave loop as a third amplitude of a third sample point of the second cardiac electrical signal.

### Example 6

The medical device of Example 5, wherein the processing circuitry is further configured to identify the third sample point of the second cardiac electrical signal at a common sample time as one of the first sample point of the first cardiac electrical signal or the second sample point of the first cardiac electrical signal.

### Example 7

The medical device of any one of Examples 1-6, wherein the processing circuit is further configured to derive the T-wave loop in at least two dimensions from a first cardiac electrical signal and a second cardiac electrical signal of the up to two cardiac electrical signals by determining a first coordinate in a first dimension and a second coordinate in a second dimension of each point of a plurality of points of the T-wave loop by: determining the first coordinate as a first amplitude of a first sample point of the first cardiac electrical signal; and determining a second coordinate as a second amplitude of a second sample point of the second cardiac electrical signal.

### Example 8

The medical device of Example 7, wherein the processing circuitry is further configured to derive the T-wave loop in three dimensions from the first cardiac electrical signal and the second cardiac electrical signal of the up to two cardiac electrical signals by determining a third coordinate of each point of the plurality of points of the T-wave loop by determining a third amplitude from a combination of the first amplitude and the second amplitude.

### Example 9

The medical device of any of Examples 1-8, wherein the processing circuitry is further configured to determine the repolarization measurement by determining a T-wave vector in the at least two dimensions from the T-wave loop.

### Example 10

The medical device of Example 9, wherein the processing circuity is further configured to determine the change in the repolarization measurement by determining an angle between the T-wave vector and a previously determined T-wave vector.

### Example 11

The medical device of any one of Examples 9 or 10, wherein the processing circuity is further configured to: determine an angle between the T-wave vector and an axis of a coordinate system corresponding to the at least two dimensions of the T-wave loop; and determine the change in the repolarization measurement by determining a difference between the angle and a previously determined angle between a previously determined T-wave vector and the axis of the coordinate system.

### Example 12

The medical device of any of claims 1-11, wherein the processing circuitry is further configured to determine the repolarization measurement by determining at least one of: an area of the T-wave loop; an area of a two-dimensional projection of the T-wave loop; a distance from a first point of the T-wave loop to a second point of the T-wave loop; a distance from an origin of a coordinate system corresponding to the at least two dimensions of the T-wave loop to a furthest point of the T-wave loop; a centroid of the T-wave loop; or a length of a perimeter of the T-wave loop.

### Example 13

The medical device of any of Example s 1-12, wherein the processing circuitry is further configured to determine the metric by a spectral analysis of frequencies of the changes in the repolarization measurements over time.

### Example 14

The medical device of any of Examples 1-12, wherein the processing circuitry is further configured to determine the metric by an amplitude analysis of the changes in the repolarization measurement over time.

### Example 15

The medical device of any of Examples 1-14, further comprising a therapy delivery circuit configured to deliver or adjust a cardiac electrical stimulation therapy in response to the metric meeting the risk threshold.

### Example 16

The medical device of any of Examples 1-15, wherein the processing circuitry is further configured to receive a first cardiac electrical signal of the up to two cardiac electrical signals from a first sensing electrode vector in a horizontal plane of a patient.

### Example 17

The medical device of any of Examples 1-16 wherein the processing circuitry is further configured to receive a second cardiac electrical signal of the up to two cardiac electrical signal from a second sensing electrode vector that is orthogonal to the first sensing electrode vector.

### Example 18

A method performed by a medical device, the method comprising: receiving up to two cardiac electrical signals by processing circuitry of the medical device; for each of a plurality of cardiac cycles of the received cardiac electrical signal(s) consisting of the up to the two cardiac electrical signals: deriving a T-wave loop in at least two dimensions; determining a repolarization measurement representative of the T-wave loop; and determining a change in the repolarization measurement from a previously determined repolarization measurement; determining a metric of the determined changes in the repolarization measurements; determining that the metric meets a risk threshold associated with a cardiac event; and transmitting a risk notification in response to the metric meeting the risk threshold.

### Example 19

The method of Example 18, further comprising deriving the T-wave loop in at least two dimensions from a first cardiac electrical signal of the up to two cardiac electrical signals by determining a first coordinate in a first dimension and a second coordinate in a second dimension of each point of a plurality of points of the T-wave loop by: determining the first coordinate as a first amplitude of a first sample point of the first cardiac electrical signal; and determining the second coordinate as a second amplitude of a second sample point of the first cardiac electrical signal, the second sample point offset by a first time interval from the first sample point.

### Example 20

The method of Example 19, further comprising deriving the T-wave loop in three dimensions from the first cardiac electrical signal by determining a third coordinate in a third dimension of each point of the plurality of points of the T-wave loop as a third amplitude of a third sample point of the first cardiac electrical signal, the third sample point offset by a second time interval from the first sample point.

### Example 21

The method of Example 20 further comprising determining the third amplitude of the third sample point offset by the second time interval from the first sample point where the second time interval is different than the first time interval

### Example 22

The method of any of Examples 19-21, further comprising deriving the T-wave loop in three dimensions from the first cardiac electrical signal and a second cardiac electrical signal of the up to two cardiac electrical signals by determining a third coordinate in a third dimension of each point of the plurality of points of the T-wave loop as a third amplitude of a third sample point of the second cardiac electrical signal.

### Example 23

The method of Example 22, further comprising identifying the third sample point of the second cardiac electrical signal at a common sample time as one of the first sample point of the first cardiac electrical signal or the second sample point of the first cardiac electrical signal.

### Example 24

The method of any of Example 18-23, further comprising deriving the T-wave loop in at least two dimensions from a first cardiac electrical signal and a second cardiac electrical signal of the up to two cardiac electrical signals by determining a first coordinate in a first dimension and a second coordinate in a second dimension of each point of a plurality of points of the T-wave loop by: determining the first coordinate as a first amplitude of a first sample point of the first cardiac electrical signal; and determining a second coordinate as a second amplitude of a second sample point of the second cardiac electrical signal.

### Example 25

The method of Example 24, wherein the processing circuitry is further configured to derive the T-wave loop in three dimensions from the first cardiac electrical signal and the second cardiac electrical signal of the up to two cardiac electrical signals by determining a third coordinate of each point of the plurality of points of the T-wave loop by determining a third amplitude from a combination of the first amplitude and the second amplitude.

### Example 26

The method of any of Examples 18-25, further comprising determining the repolarization measurement by determining a T-wave vector in the at least two dimensions from the T-wave loop.

### Example 27

The method of Example 26, further comprising determining the change in the repolarization measurement by determining an angle between the T-wave vector and a previously determined T-wave vector.

### Example 28

The method of any of Examples 26 or 27, further comprising: determining an angle between the T-wave vector and an axis of a coordinate system corresponding to the at least two dimensions of the T-wave loop; and determining the change in the repolarization measurement by determining a difference between the angle and a previously determined angle between a previously determined T-wave vector and the axis of the coordinate system.

### Example 29

The method of any of Examples 18-28, further comprising determining the repolarization measurement by determining at least one of: an area of the T-wave loop; an area of a two-dimensional projection of the T-wave loop; a distance from a first point of the T-wave loop to a second point of the T-wave loop; a distance from an origin of a coordinate system corresponding to the at least two dimensions of the T-wave loop to a furthest point of the T-wave loop; a centroid of the T-wave loop; or a length of a perimeter of the T-wave loop.

### Example 30

The method of any of Examples 18-29, further comprising determining the metric by a spectral analysis of frequencies of the changes in the repolarization measurements over time.

### Example 31

The method of any of Examples 18-30, further comprising determining the metric by an amplitude analysis of the changes in the repolarization measurement over time.

### Example 32

The method of any of Examples 18-31, further comprising delivering or adjusting a cardiac electrical stimulation therapy in response to the metric meeting the risk threshold.

### Example 33

The method of any of Examples 18-32, further comprising receiving a first cardiac electrical signal of the up to two cardiac electrical signals from a first sensing electrode vector in a horizontal plane of a patient.

### Example 34

The method of any of Examples 18-33 further comprising receiving a second cardiac electrical signal of the up to two cardiac electrical signal from a second sensing electrode vector that is orthogonal to the first sensing electrode vector.

### Example 35

A non-transitory, computer readable medium storing a set of instructions that, when executed by a control circuit of a medical device, cause the medical device to: receive up to two cardiac electrical signals; for each of a plurality of cardiac cycles of the received cardiac electrical signal(s) consisting of the up to the two cardiac electrical signals: derive a T-wave loop in at least two dimensions; determine a repolarization measurement representative of the T-wave loop; and determine a change in the repolarization measurement from a previously determined repolarization measurement; determine a metric of the determined changes in the repolarization measurements; determine that the metric meets a risk threshold associated with a cardiac event; and transmit a risk notification in response to the metric meeting the risk threshold.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device, comprising:
processing circuitry configured to:
receive up to two cardiac electrical signals;
for each of a plurality of cardiac cycles of the received cardiac electrical signal(s) consisting of the up to two cardiac electrical signals:
derive a T-wave loop in at least two dimensions;
determine a repolarization measurement representative of the T-wave loop; and
determine a change in the repolarization measurement from a previously determined repolarization measurement;
determine a metric of the determined changes in the repolarization measurements; and
determine that the metric meets a risk threshold associated with a cardiac event; and
a telemetry circuit configured to transmit a risk notification in response to the metric meeting the risk threshold.

2. The medical device of claim 1, wherein the processing circuitry is further configured to derive the T-wave loop in at least two dimensions from a first cardiac electrical signal of the up to two cardiac electrical signals by determining a first coordinate in a first dimension and a second coordinate in a second dimension of each point of a plurality of points of the T-wave loop by:
determining the first coordinate as a first amplitude of a first sample point of the first cardiac electrical signal; and
determining the second coordinate as a second amplitude of a second sample point of the first cardiac electrical signal, the second sample point offset by a first time interval from the first sample point.

3. The medical device of claim 2, wherein the processing circuitry is further configured to derive the T-wave loop in three dimensions from the first cardiac electrical signal by determining a third coordinate in a third dimension of each point of the plurality of points of the T-wave loop as a third amplitude of a third sample point of the first cardiac electrical signal, the third sample point offset by a second time interval from the first sample point.

4. The medical device of claim 3, wherein the processing circuitry is further configured to determine the third amplitude of the third sample point offset by the second time interval from the first sample point where the second time interval is different than the first time interval.

5. The medical device of claim 2, wherein the processing circuitry is further configured to derive the T-wave loop in three dimensions from the first cardiac electrical signal and a second cardiac electrical signal of the up to two cardiac electrical signals by determining a third coordinate in a third dimension of each point of the plurality of points of the T-wave loop as a third amplitude of a third sample point of the second cardiac electrical signal.

6. The medical device of claim 5, wherein the processing circuitry is further configured to identify the third sample point of the second cardiac electrical signal at a common sample time as one of the first sample point of the first cardiac electrical signal or the second sample point of the first cardiac electrical signal.

7. The medical device of claim 1, wherein the processing circuit is further configured to derive the T-wave loop in at least two dimensions from a first cardiac electrical signal and a second cardiac electrical signal of the up to two cardiac electrical signals by determining a first coordinate in a first dimension and a second coordinate in a second dimension of each point of a plurality of points of the T-wave loop by:
determining the first coordinate as a first amplitude of a first sample point of the first cardiac electrical signal;
determining a second coordinate as a second amplitude of a second sample point of the second cardiac electrical signal; and
determining a third coordinate of each point of the plurality of points of the T-wave loop by determining a third amplitude from a combination of the first amplitude and the second amplitude.

8. (canceled)

9. The medical device of claim 1, wherein the processing circuitry is further configured to:
determine the repolarization measurement by determining a T-wave vector in the at least two dimensions from the T-wave loop; and
determine the change in the repolarization measurement by determining at least an angle between the T-wave vector and a previously determined T-wave vector.

10. (canceled)

11. (canceled)

12. The medical device of claim 1, wherein the processing circuitry is further configured to determine the repolarization measurement by determining at least one of:
an area of the T-wave loop;
an area of a two-dimensional projection of the T-wave loop;
a distance from a first point of the T-wave loop to a second point of the T-wave loop;
a distance from an origin of a coordinate system corresponding to the at least two dimensions of the T-wave loop to a furthest point of the T-wave loop;
a centroid of the T-wave loop; or
a length of a perimeter of the T-wave loop.

13. The medical device of claim 1, wherein the processing circuitry is further configured to determine the metric by one or more of a spectral analysis of frequencies of the changes in the repolarization measurements over time or an amplitude analysis of the changes in the repolarization measurement over time.

14. The medical device of claim 1, further comprising a therapy delivery circuit configured to deliver or adjust a cardiac electrical stimulation therapy in response to the metric meeting the risk threshold.

15. (canceled)

16. A method comprising:
receiving up to two cardiac electrical signals;
for each of a plurality of cardiac cycles of the received cardiac electrical signal(s) consisting of the up to two cardiac electrical signals:
deriving a T-wave loop in at least two dimensions;
determining a repolarization measurement representative of the T-wave loop; and
determining a change in the repolarization measurement from a previously determined repolarization measurement;
determining a metric of the determined changes in the repolarization measurements;
determining that the metric meets a risk threshold associated with a cardiac event; and
transmitting a risk notification in response to the metric meeting the risk threshold.

17. The method of claim 16, further comprising deriving the T-wave loop in at least two dimensions from a first cardiac electrical signal of the up to two cardiac electrical signals by determining a first coordinate in a first dimension and a second coordinate in a second dimension of each point of a plurality of points of the T-wave loop by:
determining the first coordinate as a first amplitude of a first sample point of the first cardiac electrical signal; and
determining the second coordinate as a second amplitude of a second sample point of the first cardiac electrical signal, the second sample point offset by a first time interval from the first sample point.

18. The method of claim 17 further comprising deriving the T-wave loop in three dimensions from the first cardiac electrical signal by:
determining a third coordinate in a third dimension of each point of the plurality of points of the T-wave loop as a third amplitude of a third sample point of the first cardiac electrical signal, the third sample point offset by a second time interval from the first sample point, the second time interval being different than the first time interval.

19. The method of claim 17 further comprising deriving the T-wave loop in three dimensions from the first cardiac electrical signal and a second cardiac electrical signal of the up to two cardiac electrical signals by determining a third coordinate in a third dimension of each point of the plurality of points of the T-wave loop as a third amplitude of a third sample point of the second cardiac electrical signal.

20. The method of claim 16 further comprising deriving the T-wave loop in at least two dimensions from a first cardiac electrical signal and a second cardiac electrical signal of the up to two cardiac electrical signals by determining a first coordinate in a first dimension and a second coordinate in a second dimension of each point of a plurality of points of the T-wave loop by:
determining the first coordinate as a first amplitude of a first sample point of the first cardiac electrical signal;
determining a second coordinate as a second amplitude of a second sample point of the second cardiac electrical signal;
determining a third coordinate of each point of the plurality of points of the T-wave loop by determining a third amplitude from a combination of the first amplitude and the second amplitude.

21. The method of claim 16 further comprising:
determining the repolarization measurement by determining a T-wave vector in the at least two dimensions from the T-wave loop; and
determining the change in the repolarization measurement by determining at least an angle between the T-wave vector and a previously determined T-wave vector.

22. The method of claim 16 further comprising determining the repolarization measurement by determining at least one of:
an area of the T-wave loop;
an area of a two-dimensional projection of the T-wave loop;
a distance from a first point of the T-wave loop to a second point of the T-wave loop;
a distance from an origin of a coordinate system corresponding to the at least two dimensions of the T-wave loop to a furthest point of the T-wave loop;
a centroid of the T-wave loop; or
a length of a perimeter of the T-wave loop.

23. The method of claim 16 further comprising determining the metric by one or more of a spectral analysis of frequencies of the changes in the repolarization measurements over time or an amplitude analysis of the changes in the repolarization measurement over time.

24. The method of claim 16 further comprising delivering or adjusting a cardiac electrical stimulation therapy in response to the metric meeting the risk threshold.

25. A non-transitory computer-readable medium storing instructions that, when executed by processing circuitry of a medical device, cause the medical device to:
receive up to two cardiac electrical signals:
for each of a plurality of cardiac cycles of the received cardiac electrical signal(s) consisting of the up to two cardiac electrical signals:
derive a T-wave loop in at least two dimensions;
determine a repolarization measurement representative of the T-wave loop; and
determine a change in the repolarization measurement from a previously determined repolarization measurement;
determine a metric of the determined changes in the repolarization measurements;
determine that the metric meets a risk threshold associated with a cardiac event; and
in response to the metric meeting the risk threshold:
deliver or adjust a cardiac electrical stimulation therapy; or
adjust a cardiac electrical stimulation therapy that is being delivered by the medical device.
